(19) United States
(12) Patent Application Publication
(10) Pub. No.: US 2019/0076577 A1
(43) Pub. Date: Mar. 14, 2019

(54) COMPOSITE MEDICAL GRAFTS AND METHODS OF USE AND MANUFACTURE

(71) Applicant: AlloSource, Centennial, CO (US)

(72) Inventors: Peter J. Stevens, N. Richland Hills, TX (US); Reginald Stilwell, Parker, CO (US); Matthew Southard, Denver, CO (US); Adrian C. Samaniego, Highlands Ranch, CO (US); Charles Manuele, Aurora, CO (US)

(73) Assignee: AlloSource, Centennial, CO (US)

(21) Appl. No.: 16/085,476

(22) PCT Filed: Mar. 16, 2017
(86) PCT No.: PCT/US2017/022703

§ 371 Date: Sep. 14, 2018

Related U.S. Application Data
(60) Provisional application No. 62/310,375, filed on Mar. 18, 2016.

(51) Int. Cl. A61L 27/38; A61L 27/36; A61L 27/56; A61L 27/22; A61L 27/18; A61L 27/20; A61F 2/28; B33Y 80/00

(52) U.S. Cl. CPC A61L 27/3847; B33Y 70/00; A61L 27/56; A61L 27/3865; A61L 27/386; A61L 27/3852; A61L 27/3604; A61L 27/225; A61L 27/3654; A61L 27/3817; A61L 27/3821; A61L 27/18; A61L 27/20; A61L 27/367; A61L 27/3826; A61F 2/28; B33Y 80/00; A61L 27/365

(57) ABSTRACT

Provided in this disclosure are various composite grafts having a trabecular synthetic scaffold with voids defined in at least a portion of the scaffold and a biological component positioned in at least some of the voids of the scaffold. The grafts may be osteogenic, chondrogenic, osteochondrogenic, or vulnerary in nature. Also provided are methods of using the composite grafts to treat a tissue defect in a subject. Methods of manufacturing are also provided. Grafts are manufactured by additive manufacturing. Agitation may be used to combine composite grafts with additional biological component.

### CROSS-REFERENCE TO RELATED APPLICATIONS

This application claims benefit of priority of U.S. Provisional Application No. 62/310,375, filed Mar. 18, 2016, which is incorporated herein by reference in its entirety.

### BACKGROUND

Human tissue compositions, such as bone, cartilage, muscle, and skin, have been used for many years in various reconstructive surgical procedures, including treatments for certain medical conditions and tissue defects.

While autografts use tissue previously recovered from the individual who will receive the graft, allografts use tissue recovered from a donor other than the recipient. Allograft tissue is often taken from deceased donors that have donated their tissue so that it can be used to treat individuals with medical needs such as trauma patients or cancer patients who lose tissue due to disease progression or surgery. Such tissues represent a gift from the donor or the donor family to enhance the quality of life for other people.

Replicating the structure and function of human tissue in an implantable graft is a challenge as it requires a carefully-created blend of multiple components. Known methods for manufacturing tissue grafts offer limited manipulation of graft characteristics.

Hence, although existing reconstructive surgical techniques and tissue graft compositions and methods provide real benefits to patients in need thereof, still further improvements are desirable. Embodiments of the present disclosure provide solutions to at least some of these outstanding needs.

### BRIEF SUMMARY

In one aspect, provided is a composite graft that has a synthetic scaffold with a bioresorbable polymer configured in a trabecular structure, the trabecular structure having voids defined in at least a portion of the synthetic scaffold; and a biological component positioned in at least some of the voids of the synthetic scaffold, the biological component held into place within the voids as a result of friction present between the biological component and the synthetic scaffold. In some instances, the synthetic scaffold comprises an anatomical shape resembling at least one of: a whole bone or a portion thereof comprising at least 10% of the whole bone and retaining at least some of the anatomical shape of the whole bone, a whole muscle or a portion thereof comprising at least 10% of the whole muscle and retaining at least some of the anatomical shape of the whole muscle, a portion of cartilage, or a portion of skin, or wherein the synthetic scaffold has a volume of 1 cm³ or greater.

In some instances, the synthetic scaffold may be an anatomical shape resembling at least one of a whole bone or a portion thereof. In some instances, the synthetic scaffold may be at least 10% of the whole bone and retaining at least some of the anatomical shape of the whole bone. In some instances, the synthetic scaffold may be a whole muscle or a portion thereof comprising at least 10% of the whole muscle and retaining at least some of the anatomical shape of the whole muscle, a portion of cartilage, or a portion of skin, wherein the synthetic scaffold has a volume of 1 cm³ or greater.

In another aspect, provided is a method of treating a tissue defect in a subject, the method including administering to the subject a composite graft as described above at the tissue defect site of the subject.

In another aspect, provided is a method of manufacturing the composite graft described above, the method including providing a synthetic substrate that includes a bioresorbable polymer; providing the biological component; and forming the composite graft using an additive manufacturing process, wherein the biological component is positioned in at least some of the voids of the synthetic scaffold, and wherein at least some of the biological component is frictionally held into place within the voids.

In another aspect, provided is a method of manufacturing composite grafts as described above (and elsewhere in this disclosure), the method including providing a synthetic substrate that includes a bioresorbable polymer; providing a first biological component; forming a partial composite graft using an additive manufacturing process, wherein the first biological component is positioned in at least some of the voids of the synthetic scaffold; providing a second biological component; and agitating the partial composite graft with the second biological component in a processing vessel to position at least a portion of the second biological component in at least some of the voids in the synthetic scaffold, at least a portion of the first biological component, the second biological component, or both, frictionally held into place within the voids, thereby forming the composite graft. In some instances, the combining includes placing the partial composite graft and the biological component into the processing vessel; and applying resonant acoustic energy to the processing vessel, the resonant acoustic energy vibrating the processing vessel such that at least a portion of the second biological component is positioned within at least some of the voids defined in the synthetic scaffold and is frictionally held into place within the voids.

In one aspect, provided is a composite graft that has a scaffold with a trabecular structure, the trabecular structure having voids defined in at least a portion of the synthetic scaffold; and a biological component positioned in at least some of the voids of the synthetic scaffold.

In another aspect, provided is a method of treating a tissue defect in a subject, the method including the step of administering to the subject the composite graft of any of the composite grafts described above (or described elsewhere in this disclosure) at the tissue defect site of the subject. In some instances, the tissue defect may be a degenerated or damaged spinal disc, a bone defect, an oral defect, a maxillofacial defect, a cartilage defect, an osteochondral defect, a muscle defect, or a skin defect. In some instances, the composite graft may be contacted with a saline solution, an antibiotic, blood, platelet rich plasma, or a combination of any thereof, prior to administering to the subject.

In another aspect, provided is a method of manufacturing the composite graft of one of composite grafts described above, the method including the steps of (a) providing a synthetic substrate; (b) providing a biological component; and (c) forming the composite graft using an additive manufacturing process, wherein the biological component is positioned in at least some of the voids of the synthetic scaffold.

In some instances, the biological component may be mixed (combined) with the synthetic substrate prior to the additive manufacturing process. The resulting mixture may then be used to construct the composite graft during the additive manufacturing process.

In another aspect, provided is a method of manufacturing the composite grafts as described above, the method including the steps of (a) providing a synthetic substrate; (b) providing a first biological component; (c) forming a partial composite graft using an additive manufacturing process, wherein the first biological component is positioned in at least some of the voids of the synthetic scaffold; (d) providing a second biological component; (e) agitating the partial composite graft with the second biological component in a processing vessel to position at least a portion of the second biological component in at least some of the voids in the synthetic scaffold thereby forming the composite implant.

In some instances, the agitating step of the manufacturing methods includes the steps of (i) placing the partial composite graft and the biological component into the processing vessel; and (ii) applying resonant acoustic energy to the processing vessel, the resonant acoustic energy vibrating the processing vessel such that at least a portion of the second biological component is positioned within at least some of the voids defined in the synthetic scaffold. In some instances, the resonant acoustic energy may be applied to the processing vessel for a period of time between 2 minutes and 4.5 hours. In some instances, the resonant acoustic energy may be applied in one or more intervals, each interval being a period of time.

In another aspect, provided is a system for manufacturing any of the composite grafts described above, the system comprising an additive manufacturing device. In some instances, the system includes a processing vessel and an agitation mechanism. In some instances, the agitation mechanism may include a shaker, a mechanical impeller mixer, a ultrasonic mixer, a sonicator, or other high intensity mixing device.

### BRIEF DESCRIPTION OF THE DRAWINGS

These figures are intended to be illustrative, not limiting. Although the aspects of the disclosure are generally described in the context of these figures, it should be understood that it is not intended to limit the scope of the disclosure to these particular aspects.

FIG. 1A-1D show exemplary scaffold and graft configurations according to some aspects of the disclosure.

FIG. 2A-2J show exemplary bone graft configurations according to some aspects of the disclosure.

FIG. 3A-3C show exemplary cartilage graft configurations according to some aspects of the disclosure.

FIG. 4A shows an exemplary cartilage graft configuration according to some aspects of the disclosure. FIG. 4B and FIG. 4C show exemplary osteochondral graft configurations according to some aspects of the disclosure. FIG. 4D shows exemplary cartilage and osteochondral graft configurations according to some aspects of the disclosure.

FIG. 5 shows exemplary muscle graft configurations according to some aspects of the disclosure.

FIG. 6A and FIG. 6B show exemplary sheet graft configurations according to some aspects of the disclosure.

FIG. 7 shows a flowchart of an exemplary method of treatment according to some aspects of the disclosure.

FIG. 8 shows a schematic of an exemplary system for manufacturing the composite grafts according to some aspects of the disclosure.

FIG. 9 shows an a flowchart of an exemplary method for manufacturing the composite grafts according to some aspects of the disclosure.

FIG. 10A and FIG. 10B show exemplary methods for manufacturing the composite grafts according to some aspects of the disclosure.

### DETAILED DESCRIPTION

### I. Introduction

This disclosure provides products, methods, and systems in the field of medical grafts and, particularly, to implantable composite grafts and methods for their manufacture and use. The composite grafts, along with the systems and methods for making and using such grafts, as disclosed herein are useful in various industries including orthopedics, reconstructive surgery, dental surgery, and cartilage replacement.

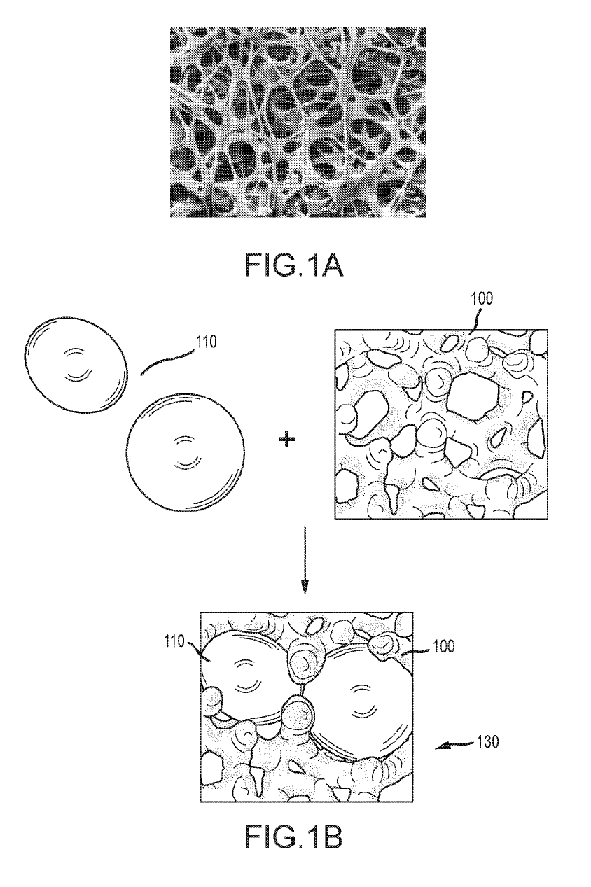

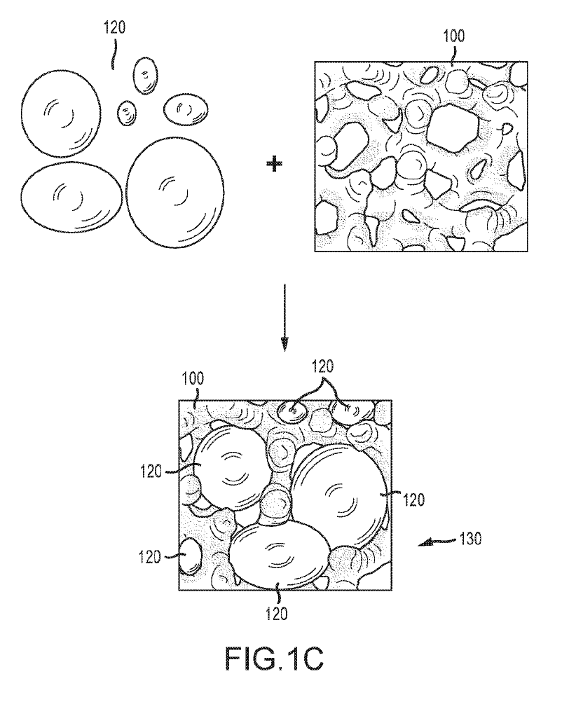

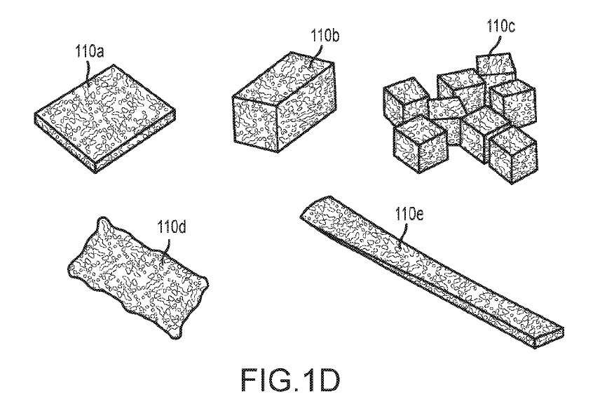

The composite grafts of the disclosure (also referred to herein as a grafts, trabecular-like grafts, among other nomenclature used) include a scaffold and biological components. The biological component of the grafts is particulate in nature, including one or more kinds of tissue, cells, or other therapeutic particles selected based on the intended use of the graft. The biological tissue component may be obtained from a deceased donor, derived from deceased donor tissue, obtained from a living donor, or derived from living donor tissue. In some instances, the biological tissue component may be recombinantly produced. The scaffold is a synthetic scaffold having a trabecular structure in which plates, rods, and struts of synthetic material form a three-dimensional network defining a plurality of voids, mimicking natural trabecular bone structure. FIG. 1A shows an example of a portion of cancellous bone 100a having a characteristic trabecular structure. The structure of cancellous bone, also referred to as spongy bone, includes plates (trabeculae) and bars (rods) of bone (calcified collagen fibers) adjacent to small, irregular cavities (voids), having the appearance of a sponge-like, open-celled network. The structure may appear to arranged in a haphazard manner, but it is organized to provide structural strength similar to braces or trusses that are used to support a building or bridge. The the voids in the synthetic scaffold are of sufficient size to admit and hold (retain) the biological component particles. The biological component and synthetic scaffold are combined such that the biological component particles are positioned within the voids of the synthetic scaffold. For illustrative purposes, FIG. 1B shows an exemplary synthetic scaffold 100 and exemplary biological component particles 110 that are uniform (or relatively uniform) in shape and size. When combined to form the composite graft 130, the biological component particles 110 are positioned within the voids defined in the synthetic scaffold 100. For illustrative purposes, FIG. 1C shows an exemplary synthetic scaffold 100b and exemplary biological component particles 120 that are not uniform in size or shape. When combined to form the composite graft 130, the biological component particles 120 are positioned within the voids defined in the synthetic scaffold 100b. Some exemplary shapes of grafts 110a-110e are shown in FIG. 1D.

The composite grafts are useful for implantation into a subject having a defect site. The defect site may be degenerated or damaged spinal disc, a bone defect, an oral defect, a maxillofacial defect, a cartilage defect, an osteochondral defect, a muscle defect, or a skin defect. The composite grafts described in this disclosure can be used to replace damaged, removed, or degenerated tissue, such as bone, cartilage, muscle, and skin. The graft may contain a biological component that is therapeutic to healing the defect site such as by promoting tissue growth. In some instances, the graft may contain a biological tissue component derived from a similar tissue type as present at the implantation site or containing biological components that may be found at the implantation site or that would act to promote tissue growth at the implantation site. In some instances, the region of implantation does not have tissue similar to the biological component of the graft but may still cause therapeutic benefit. The terms patient and subject are used interchangeably in this disclosure.

When the composite grafts are implanted in a patient, the synthetic scaffold may act as a stable physical support structure at the defect site, replacing or supporting damaged, removed, or degenerated tissue, and the biological component may increase the ability of the implant to be integrated into the patient, reducing risk of rejection and encapsulation. In some cases, the grafts may be fabricated to better mimic any of natural tissue function, natural tissue appearance, or natural tissue configuration at the implantation site (also referred to as an implant site) while offering the additional stability of the synthetic scaffold. The grafts may also be customized to best suit a particular patient. It is contemplated that the combination of the synthetic component with the biological component may provide improved graft structure, stability, and function over currently known implant compositions and devices.

Traditional methods of making grafts having a synthetic scaffold and a biological component(s) generally focus on coating the surface of the scaffold with the biological component(s) (such that the biological component is “painted on”), producing scaffold with physical indentations on the surface (dimpling) to mimic the surface nanoarchitecture of human tissue, or seeding cells on a scaffold and allowing them to adhere and, in some instances, grow to populate the scaffold. In contrast, the methods and systems provided in this disclosure yield a graft having porosity in a manner similar to biological tissues and that incorporates one or more biological components within the scaffold structure itself.

The composite grafts are fabricated using an additive manufacturing process, also referred to herein as three-dimensional (3D) printing. A synthetic material is used to fabricate the synthetic scaffold. Generally, the synthetic material has a melting temperature not greater than 50° C. During the additive manufacturing process, a synthetic material is printed into the form of the synthetic scaffold using an additive manufacturing device. Printing the synthetic scaffold permits precise control over the configuration of its trabecular structure and overall shape and size. The scaffold may be printed to be uniformly trabecular or may have voids defined only in certain regions of the scaffold. In addition, the scaffold may be fabricated such that the voids defined therein are of a particular size, or range of sizes, that are particularly suitable to admit and retain the biological component particles. Further, the low melting temperature of the synthetic material permits the biological component to be combined with the scaffold during printing without negatively impacting the biological or therapeutic activity thereof. For example, proteins may denature at around 50° C.

In some instances, the grafts may be combined with another biological component using agitation. As discussed in more detail below, agitation may be used to embed an additional (second) biological component into voids defined in the scaffold.

The methods and systems for making the composite grafts disclosed herein may increase yield in the production process by providing more uniform, customized, and predictable graft products. For instance, the systems and methods disclosed herein may utilize donor tissue regardless of size and shape to produce a medical graft that is more uniform in size and composition, among other qualities.

In one aspect, provided is a composite graft comprising a synthetic scaffold, the synthetic scaffold comprising a bioresorbable polymer configured in a trabecular structure, the trabecular structure comprising voids defined in at least a portion of the synthetic scaffold; and a biological component positioned in at least some of the voids of the synthetic scaffold, the biological component held into place within the voids as a result of friction present between the biological component and the synthetic scaffold (frictionally held). In some instances, a portion of the biological component within the scaffold may be held within the voids by friction. In some instances, all the biological component within the scaffold may be held within the voids by friction. In some instances, the synthetic scaffold may comprise an anatomical shape resembling at least one of: (i) a whole bone or a portion thereof comprising at least 10% of the whole bone and retaining at least some of the anatomical shape of the whole bone, (ii) a whole muscle or a portion thereof comprising at least 10% of the whole muscle and retaining at least some of the anatomical shape of the whole muscle, (iii) a portion of cartilage, or (iv) a portion of skin. In some instances, the synthetic scaffold may comprise a volume of 1 cm³ or greater.

In some instances, the synthetic scaffold may comprise an anatomical shape resembling a whole bone or a portion thereof having at least 10% of the whole bone and retaining at least some of the anatomical shape of the whole bone. In some instances, the synthetic scaffold may comprise an anatomical shape resembling a whole muscle or a portion thereof having at least 10% of the whole muscle and retaining at least some of the anatomical shape of the whole muscle. In some instances, the synthetic scaffold may comprise an anatomical shape resembling a portion of cartilage. In some instances, the synthetic scaffold may comprise an anatomical shape resembling a portion of skin.

In some instances, in the composite graft described above, the synthetic scaffold may comprise an anatomical shape resembling at least one of a whole bone or a portion thereof. In some instances, the synthetic scaffold may be at least 10% of the whole bone and retaining at least some of the anatomical shape of the whole bone. In some instances, the synthetic scaffold may be a whole muscle or a portion thereof having at least 10% of the whole muscle and retaining at least some of the anatomical shape of the whole muscle, a portion of cartilage, or a portion of skin, wherein the synthetic scaffold has a volume of 1 cm³ or greater.

In some instances, in the composite graft described above, the biological component may comprise an osteogenic biological component, a chondrogenic biological component, a vulnerary biological component, or a combination of two or more thereof. In some instances, in the composite graft described above, the osteogenic biological component may comprise at least one of osteogenic tissue particles, osteogenic cells, or bone morphogenic proteins. In some instances, in the composite graft described above, the osteogenic cells may comprise at least one of mesenchymal stem cells, osteoblasts, or platelet rich plasma. In some instances, in the composite graft described above, the chondrogenic biological component may comprise chondrogenic tissue particles, chondrogenic cells, a chondrogenic growth factor, or a combination of two or more thereof. In some instances, in the composite graft described above, the chondrogenic cells may comprise at least one of mesenchymal stem cells or chondrocytes. In some instances, in the composite graft described above, the vulnerary biological component may comprise at least one of dermal tissue particles, muscle tissue particles, mesenchymal stem cells, keratinocytes, platelet rich plasma, dermal tissue particles seeded with mesenchymal stem cells, dermal tissue particles seeded with keratinocytes, or muscle tissue particles seeded with mesenchymal stem cells. In some instances, in the composite graft described above, the biological component may be recovered from a cadaveric donor.

In some instances, in the composite graft described above, the graft may comprise a crescent shape, a wedge shape, a cylindrical shape, a spherical shape, a cubic shape, a pyramid shape, a cone shape, or an irregular shape.

In some instances, the composite graft may comprise a biological adhesive.

In another aspect, provided is a method of treating a tissue defect in a subject, the method including administering to the subject the composite graft as described in this disclosure at the tissue defect site of the subject.

In some instances, the tissue defect may comprise a degenerated or damaged spinal disc, a bone defect, an oral defect, a maxillofacial defect, a cartilage defect, an osteochondral defect, a muscle defect, or a skin defect. In some instances, the composite graft may be contacted with a saline solution, an antibiotic, blood, platelet rich plasma, or a combination of any thereof, prior to administering to the subject.

In another aspect, provided is a method of manufacturing a composite graft as described in this disclosure, the method including providing a synthetic substrate comprising a bioresorbable polymer; providing a biological component; and forming the composite graft using an additive manufacturing process, wherein the biological component is positioned in at least some of the voids of the synthetic scaffold, and wherein at least some of the biological component is frictionally held into place within the voids. In some instances, the biological component is mixed with the synthetic substrate prior to the additive manufacturing process. In some instances, the method comprises combining at least one of the synthetic scaffold or the biological component with a biological adhesive prior to or during the additive manufacturing process. In some instances, the method comprises adding a biological adhesive onto at least one of the synthetic scaffold or the biological component during the additive manufacturing. In some instances, the composite graft may be mixed (combined) with one of a biocompatible solution or an additional biological component. In some instances, the composite graft may be mixed (combined) with at least one of a biocompatible solution or an additional biological component. In some instances, the biocompatible solution may be a buffered solution, a nutritive media, or a cryopreservation medium.

In another aspect, provided is a method of manufacturing a composite graft as described in this disclosure, the method comprising providing a synthetic substrate comprising a bioresorbable polymer; providing a first biological component; forming a partial composite graft using an additive manufacturing process, wherein the first biological component is positioned in at least some of the voids of the synthetic scaffold; providing a second biological component; and agitating the partial composite graft with the second biological component in a processing vessel to position at least a portion of the second biological component in at least some of the voids in the synthetic scaffold, at least a portion of the first biological component, the second biological component, or both, frictionally held into place within the voids, thereby forming the composite graft. In some instances, the combining comprises placing the partial composite graft and the first biological component into the processing vessel; and applying resonant acoustic energy to the processing vessel, the resonant acoustic energy vibrating the processing vessel such that at least a portion of the second biological component is positioned within at least some of the voids defined in the synthetic scaffold and is frictionally held into place within the voids. In some instances, the resonant acoustic energy may be applied to the processing vessel for a period of time between 2 minutes and 4.5 hours. In some instances, the resonant acoustic energy may be applied in one or more intervals, each interval being a period of time. In some instances, the method comprises combining at least one of the synthetic scaffold or the biological component with a biological adhesive prior to agitating. In some instances, the method comprises combining the composite graft with at least one of a biocompatible solution or an additional biological component. In some instances, the biocompatible solution may be a buffered solution, a nutritive media, or a cryopreservation medium.

In another aspect, provided is a composite graft comprising a scaffold, the scaffold comprising a trabecular structure, the trabecular structure having voids defined in at least a portion of the synthetic scaffold; and a biological component positioned in at least some of the voids of the synthetic scaffold. In some instances, the synthetic scaffold comprises a bioresorbable polymer.

In some instances, the biological component comprises an osteogenic biological component, a chondrogenic biological component, a vulnerary biological component, or a combination of two or more thereof. In some instances, the osteogenic biological component comprises osteogenic tissue particles, osteogenic cells, bone morphogenic proteins, or a combination of two or more thereof. In some instances, the osteogenic cells comprises at least one of mesenchymal stem cells, osteoblasts, or platelet rich plasma.

In some instances, the chondrogenic biological component comprises chondrogenic tissue particles, chondrogenic cells, a chondrogenic growth factor, or a combination of two or more thereof. In some instances, the chondrogenic cells comprise at least one of mesenchymal stem cells or chondrocytes.

In some instances, the vulnerary biological component comprises at least one of dermal tissue particles, muscle tissue particles, mesenchymal stem cells, keratinocytes, platelet rich plasma, dermal tissue particles seeded with mesenchymal stem cells, dermal tissue particles seeded with keratinocytes, or muscle tissue particles seeded with mesenchymal stem cells.

In some instances, the graft comprises a crescent shape, a cylindrical shape, or an irregular shape corresponding to a bone, a portion of a bone, a tissue, a portion of a tissue, or a combination of two or more thereof.

In some instances, the graft comprises a biological adhesive.

In some instances, the graft comprises a second biological component.

In another aspect, provided is a method of treating a tissue defect in a subject, the method including the step of administering to the subject a composite graft as described in this disclosureat the tissue defect site of the subject. In some instances, the tissue defect comprises a degenerated or damaged spinal disc, a bone defect, an oral defect, a maxillofacial defect, a cartilage defect, an osteochondral defect, a muscle defect, or a skin defect. In some instances, the composite graft may be contacted with a saline solution, an antibiotic, blood, platelet rich plasma, or a combination of any thereof, prior to administering to the subject.

In another aspect, provided is a method of manufacturing a composite graft as described in this disclosure, the method comprising the steps of (a) providing a synthetic substrate; (b) providing a biological component; and (c) forming the composite graft using an additive manufacturing process, wherein the biological component is positioned in at least some of the voids of the synthetic scaffold.

In some instances, the biological component may be mixed (combined) with the synthetic substrate prior to the additive manufacturing process. The resulting mixture would used to construct the composite graft during the additive manufacturing process.

In some instances, a biological adhesive may be combined with at least one of the synthetic scaffold or the biological component prior to or during the additive manufacturing process. In some instances, a biological adhesive may be added onto at least one of the synthetic scaffold or the biological component during the additive manufacturing. For example, the biological adhesive may be added as a component separate from the biological component and the synthetic substrate to the synthetic scaffold as it is being constructed. In one example, the biological adhesive may be added as a layer on top of a portion of the synthetic scaffold before a biological component is added to that portion of the synthetic scaffold during the additive manufacturing process.

In some instances, the composite graft may be combined with at least one of a biocompatible solution or an additional biological component. In some instances, the biocompatible solution may be a buffered solution, a nutritive media, or a cryopreservation medium.

In another aspect, provided is a method of manufacturing a composite graft as described in this disclosure, the method including the steps of (a) providing a synthetic substrate; (b) providing a biological component; (c) forming a partial composite graft using an additive manufacturing process, wherein the biological component is positioned in at least some of the voids of the synthetic scaffold; (d) providing a second biological component; (e) agitating the partial composite graft with the second biological component in a processing vessel to position at least a portion of the second biological component in at least some of the voids in the synthetic scaffold thereby form the composite implant. In some instances, the method may comprise combining at least one of the synthetic scaffold or the biological component with a biological adhesive prior to agitating.

In some instances, the agitating step comprises the steps of (i) placing the partial composite graft and the biological component into the processing vessel; and (ii) applying resonant acoustic energy to the processing vessel, the resonant acoustic energy vibrating the processing vessel such that at least a portion of the second biological component is positioned within at least some of the voids defined in the synthetic scaffold. In some instances, the resonant acoustic energy may be applied to the processing vessel for a period of time between 2 minutes and 4.5 hours. In some instances, the resonant acoustic energy may be applied in one or more intervals, each interval comprising a period of time.

In some instances, the composite graft may be combined with at least one of a biocompatible solution or an additional biological component. In some instances, the biocompatible solution may comprise a buffered solution, a nutritive media, or a cryopreservation medium.

In another aspect, provided is a system for manufacturing any of the composite grafts described above, the system comprising an additive manufacturing device. In some instances, the system includes a processing vessel and an agitation mechanism. In some instances, the agitation mechanism may include a shaker, a mechanical impeller mixer, a ultrasonic mixer, a sonicator, or other high intensity mixing device.

### II. Composite Grafts

The composite grafts of this disclosure are useful for implantation into a subject at a defect site. The grafts contain biological components that promote tissue regeneration, integration of the grafts at an implantation site in a subject, or both. Grafts having different compositions and configurations are suitable for implantation at different kinds of defect sites.

The composite grafts may be configured to correspond to an intended implant site. For example, the configuration of the graft will dictate the defect site at which the graft may be implanted. The grafts may have an overall shape, surface area, thickness, and/or other measurement that is compatible with the physical characteristics of an intended implant site. In some instances, the grafts may be resistant to erosion or degradation after implantation into a subject. For instance, the grafts and, more particularly, the synthetic scaffold component thereof, may not break down from normal movement or may break down very slowly over a lifetime of the patient (wear free or resistant). In another example, the grafts and, more particularly, the synthetic scaffold component thereof, may degrade or erode over a lifetime of the patient. In some instances, the synthetic scaffold component of the grafts is bioresorbable.

The composite grafts may include one type of biological tissue component or may contain a plurality of types of biological tissue components. The composite grafts may contain an osteogenic biological component, a chondrogenic biological component, a vulnerary biological component, or combinations thereof. The nature of the biological component is relevant to the use of the graft. Grafts containing an osteogenic biological component may be useful for implantation at a bone defect site to promote bone growth and integration of the graft into the bone tissue at the defect site. Grafts containing a chondrogenic biological component may be useful for implantation at a cartilage defect site to promote cartilage growth and integration of the graft into the cartilage tissue at the defect site. Grafts containing at least one of an osteogenic biological component and a chondrogenic biological component may be useful for implantation at an osteochondral defect site to promote bone growth, cartilage growth, or both, and integration of the graft into the tissue at the defect site. Grafts containing a vulnerary biological component may be useful for implantation at a muscle or skin defect site to promote tissue growth and integration of the graft into the tissue at the defect site.

The composite grafts may be configured in various shapes and sizes. In some instances, the shape and size of the grafts is determined the configuration of the synthetic scaffold. For example the synthetic scaffold may be fabricated in the desired shape and size of the graft. In some instances, the synthetic scaffold may be cut or machined to a final desired shape, size, or both. In some instances, where the synthetic scaffold of the grafts is sufficiently soft, the graft may be shaped by surgical device (such as a scalpel or other sharp device) prior to implantation. In some instances, the composite grafts may have a shape such as, for example, a cube, strip, sphere, or wedge, that may be efficiently and/or quickly manufactured and packaged. Such grafts may be cut or machined into such shapes after combination with the biological component.

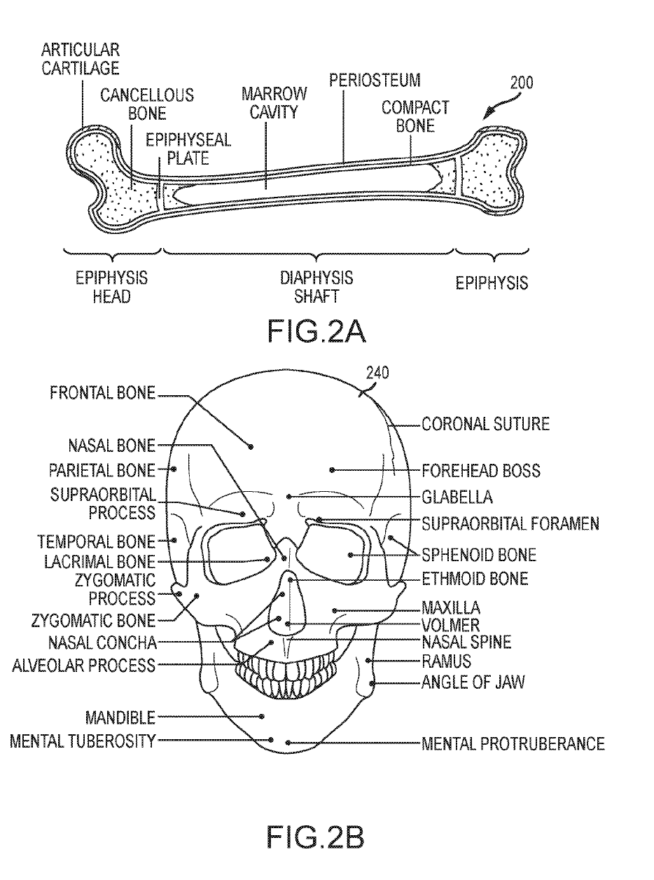

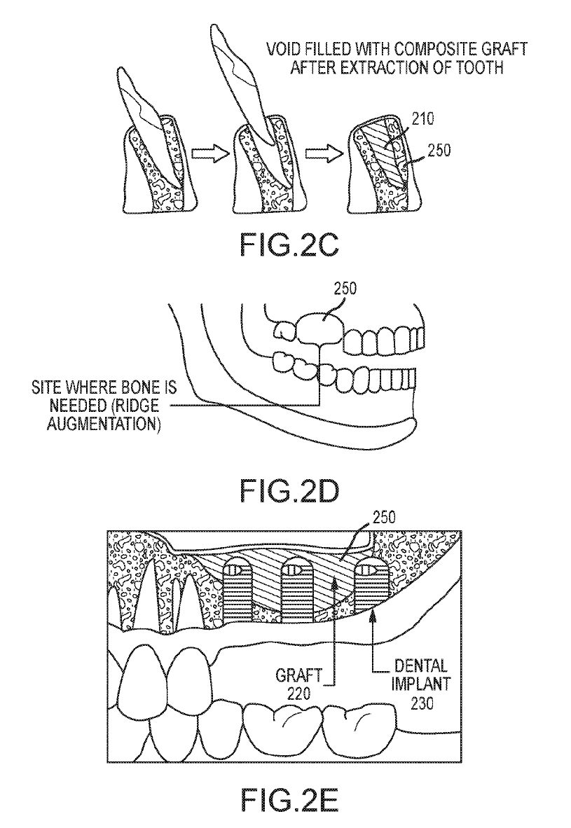

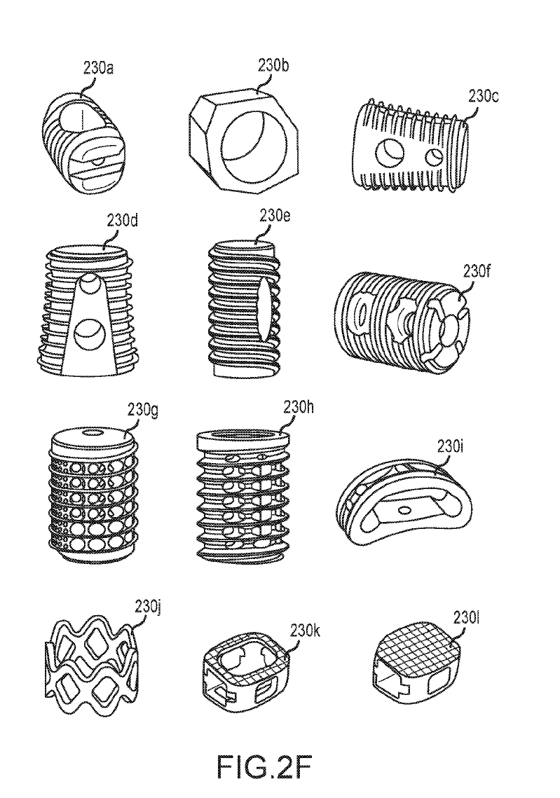

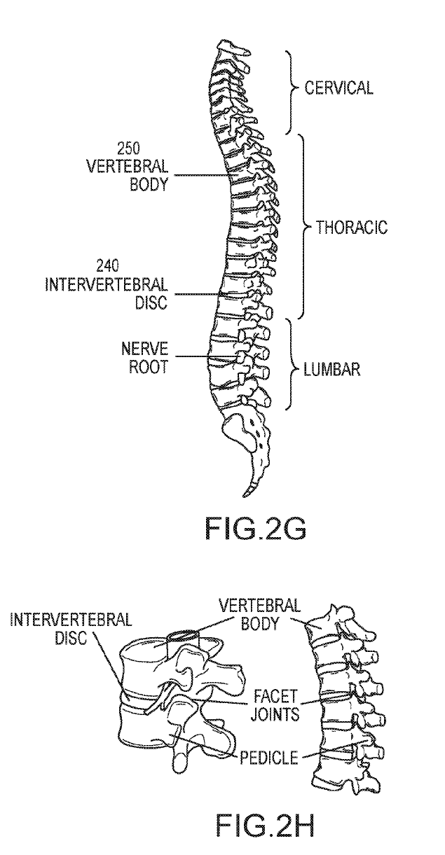

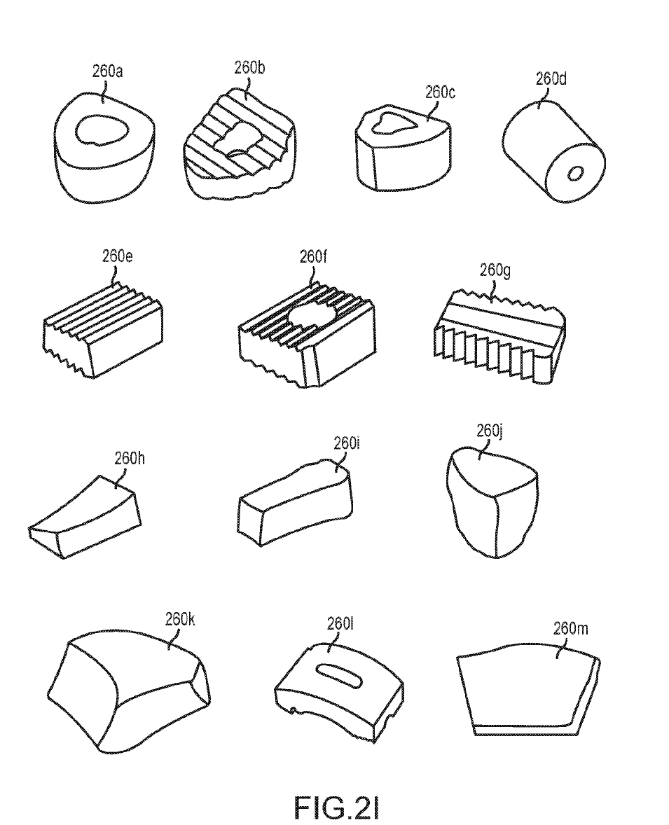

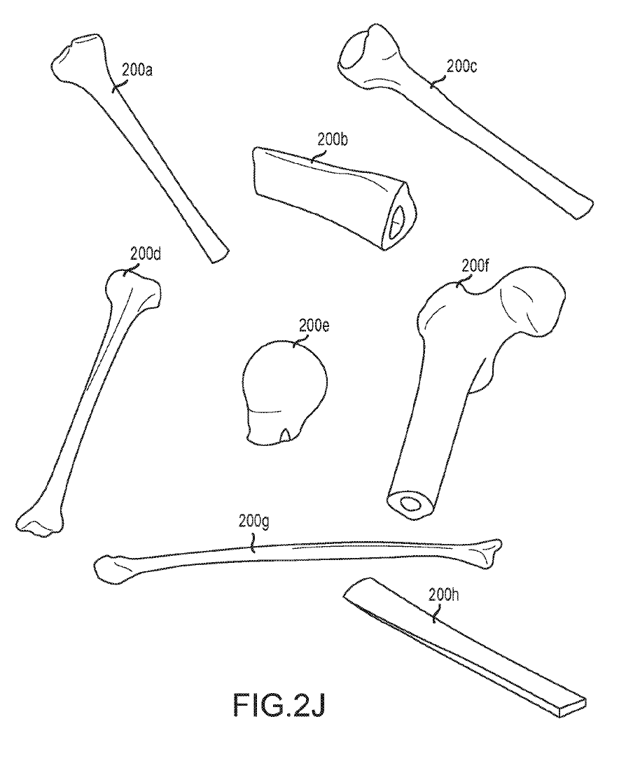

The composite grafts may be configured in the shape of a tissue found in a subject. As discussed elsewhere in this disclosure, the grafts are suitable for implantation at a defect site in a subject. The defect site may be a site within the body of the subject at which the native tissue is damaged or missing. The grafts may be implanted into such defect site to fill a void defined by the damaged or missing tissue. The grafts may be configured in the shape and size of an anatomical body part. In some instances, the grafts may have a crescent shape, a cylindrical shape, a thin sheet-like shape, an irregular shape, a shape corresponding to a muscle, or a shape corresponding to at least a portion of a long bone, a short bone, a flat bone, an irregular bone, or a vertebrae disc. A wide variety of other shapes and sizes for the grafts are contemplated. Exemplary graft configurations are shown in, or are readily apparent from, FIGS. 2A-2J, FIGS. 3A-3C, FIGS. 4A-4C, and FIGS. 5-7, as discussed further below.

In some instances, the composite grafts may be configured in the shape of a bone. In some instances, the grafts may be configured in the shape of a long bone or a portion thereof. Long bones are hard, dense bones that provide strength, structure, and mobility. A long bone has a shaft and two ends. There are also bones in the fingers that are classified as “long bones,” even though they are relatively short in length, due to the shape of the bones. For example, FIG. 2A depicts a long bone, such as a long bone found in an arm or leg, having a ephipysis head, a diaphysis shaft, and an ephiphysis. Grafts may be configured in the shape of the entire long bone or a portion thereof. By way of example, the grafts may be configured to represent 10%-80% of a long bone. For example, the graft may have an elongated cylindrical shape. In some instances, the graft may have an irregular shape configured similar to at least one end of a long bone. Depending on which portion of the long bone the graft is intended to replace, the graft may be more or less porous to mimic the degree of porosity of the native bone. For example, if the graft is configured to replace one of the ends of the long bone, which are naturally relatively porous, the graft may be relatively porous throughout its structure. In another example, if the graft is configured to replace a central portion of a long bone, it may only have porosity at the end to be adjoined to a native portion of bone and, optionally, at the opposite end. In some instances, grafts intended to be implanted at a defect in a long bone may replace portions of both the shaft and one of the ends of the bone. In such instances, the shaft portion of the graft may be less porous and, potentially, harder and less flexible, than the end portion of the graft. Exemplary shapes of grafts 200a-200h in the shape of a bone or portion thereof are shown in FIG. 2J. As discussed further elsewhere in this disclosure, such grafts may include osteogenic biological components.

FIG. 2B depicts a front view of a human skull 240. Many facial bones have an irregular shape. The composite grafts may be configured in a shape similar to any of the bones of the human skull 240, or portions thereof, as depicted in FIG. 2B. In addition to the anterior bones of the skull labeled in FIG. 2B, also contemplated herein are grafts in the shape of bones on the posterior or sides of the skull, or portions thereof, the general shape of such bones being known in the art. For example, certain bones of the skull that are not shown are the occipital bone, the mastoid protrusion, and the styloid protrusion. At least some of the grafts may be considered maxilofacial grafts. In some instances, the grafts may be configured in a shape similar to a region of the face comprising a plurality of bones. In some instances, the grafts may be configured in a shape similar to one or more of the skull bones on the side or posterior of the human skull. While FIG. 2B depicts a human skull, it is understood that grafts may be configured in the shape of bones of non-human animal skulls as well. It is also understood that composite grafts may be configured in the shape of any irregular bone in a subject's body. As discussed further elsewhere in this disclosure, such grafts may include osteogenic biological components.

FIG. 2C-2E depict various oral defects, maxilofacial defects, and appropriate grafts. In some instances, the composite graft 210 may be implanted at an implant site 250 at the site of a tooth extraction as depicted in FIG. 2C. As shown in FIG. 2D and FIG. 2E, a portion of the upper ridge, or of the jaw (not shown), may be missing or damaged in some subjects. In some instances, composite grafts may be configured in an irregular shape, such as graft 220, so as to fit into an implant site 250 that is the site of the missing or damaged bone areas of the jaw or upper ridge. In some instances, a composite graft may be configured as a dental grafts, such as graft 230 as shown in FIGS. 2E-2F. Such grafts may be configured to receive an artificial replacement tooth (such as via an internal threaded cavity formed within the graft). In some instances, the graft may include an artificial replacement tooth. As discussed further elsewhere in this disclosure, such grafts may include osteogenic biological components.

In another example, the composite grafts may be configured in a shape suitable for an intervertebral disc graft. Graft shapes include cylindrical shapes, conical shapes, box shapes, rectangular shapes, rounded box shapes, rounded rectangular shapes, and wedge shapes. Exemplary shapes of grafts 230a-230l are shown in FIG. 2F and 260a-260m in FIG. 2I. Grafts may optionally include an internal cavity formed in a central portion of the graft (as shown in FIG. 2F). In some instances, grafts may have a cage-like structure having continuous or discontinuous exterior walls defining an internal cavity. Intervertebral disc (IVD) grafts, also referred to as cages, are used for spinal fusions. See general discussion of such cages in Steffen, T. et al., Eur. Spine J. 9(Suppl. 1):S89-S94 (2000). As shown in FIG. 2G, an intervertebral disc 240 has upper and lower flat/planar surfaces (IVD contact surfaces) that contact the flat/planar surfaces of the vertebral bodies 250 (VB contact surfaces) above and below the intervertebral disc, respectively. The surface area of the IVD contact surfaces of a intervertebral disc 240 is proportional to the surface area of the VB contact surfaces of the vertebral bodies 250 adjacent to the intervertebral disc 240 (above and below it). As the vertebral bodies 250 progressively increase in size down the length of the spine, the VB contact surfaces and the IVD contact surfaces progressively increase in size as well as does the height of the height of the invertebrate discs 240. Grafts of the disclosure may be used to replace an intervertebral disc 240 between two vertebral bodies 250. Grafts intended for different regions of the spine (cervical, thoracic, lumbar) may have different dimensions. In some instances, grafts may have one or more continuous contact surfaces. An example of such a graft is graft 2301 as shown in FIG. 2F. In some instances, the grafts may have one or more discontinuous contact surfaces, the contact surfaces being defined by an outer periphery. Examples of such grafts include, but are not limited to, grafts 230b, 230e, 230i, and 230k as shown in FIG. 2F. In some instances, the intervertebral disc grafts provided may have a surface area in the range of 120 mm² to 200 mm². In some instances, the intervertebral disc grafts provided may have a height (thickness) in the range of 5 mm to 21 mm. In one example, grafts for the cervical region of the spine may have a height in the range of 5 mm to 7 mm. In another example, grafts for the thoracic and lumbar regions of the spine may have a height in the range of 7 mm to 21 mm.

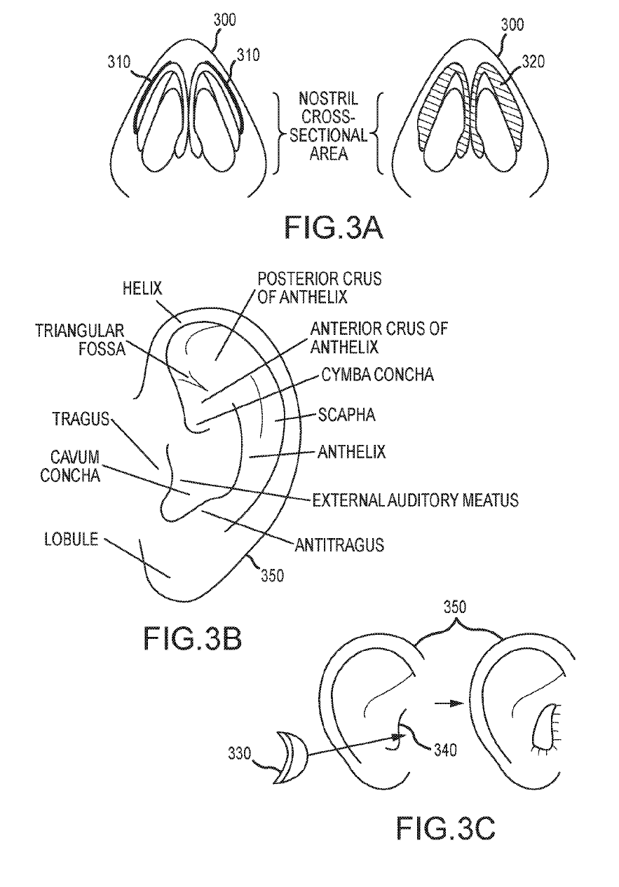

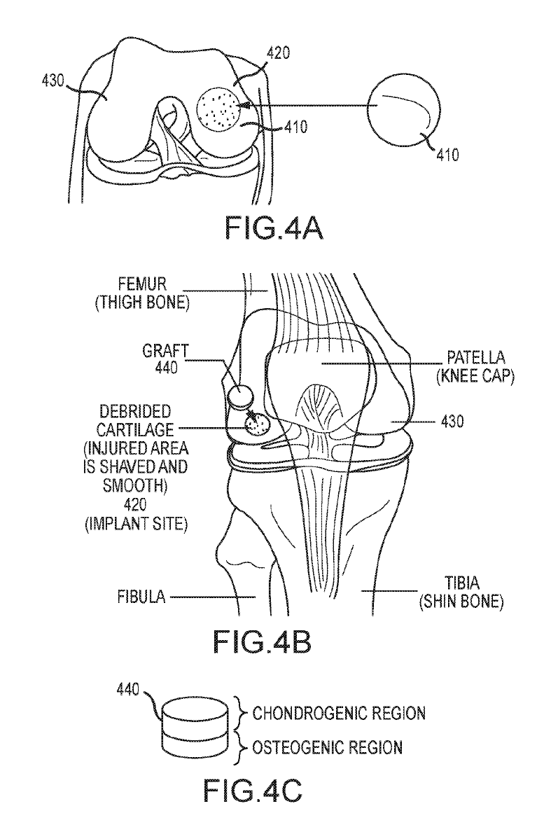

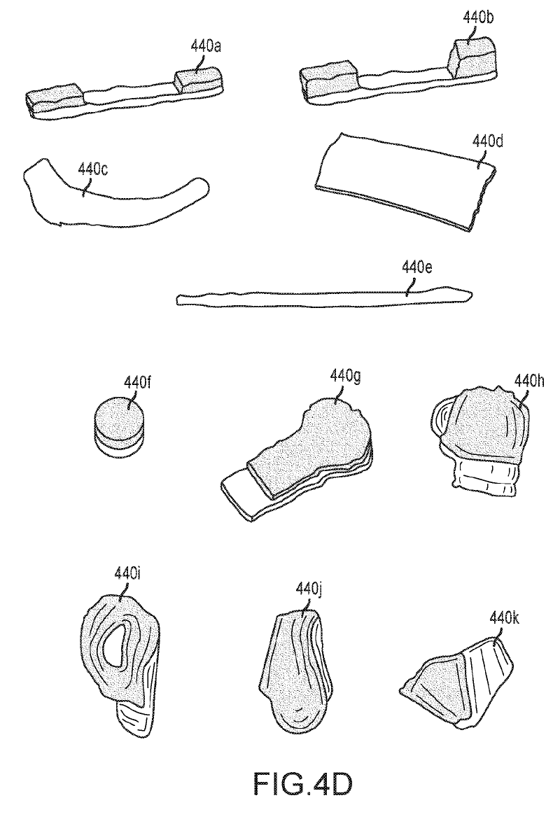

In some instances, the composite grafts may be configured in the shape of a portion of cartilage. Cartilage is a connective tissue found in many areas of an animal's body, including the joints between bones, the rib cage, the ear, the nose, the bronchial tubes and the intervertebral discs. Exemplary composite grafts to replace cartilage are shown in, or are readily apparent from, FIGS. 3A-3C and FIGS. 4A-4C. In some instances, composite grafts may have an irregular configuration suitable as a nasal graft to replace cartilage in the nose 300. Exemplary nasal grafts 310 and 320 are depicted in FIG. 3A. In some instances, composite grafts may have an irregular configuration suitable as an ear graft. FIG. 3B depicts a human ear 350 in which various parts thereof are labeled. Composite grafts may be configured in the shape of any portion of the ear or an entire ear. In one example, composite grafts may be configured in the shape of a cresent, mimicing the shape of the tragus portion of a human ear 350, such as graft 330 depicted in FIG. 3C, which is implanted at impant site 340. It is understood that non-human ears may have similar or different external components and configurations that are also contemplated as acceptable graft configurations.

In some instances, the composite grafts may be configured in the shape of a cartilage patch or an osteochondral plug. Such grafts may be suitable for implantation at various sites, including at a knee joint 430 as depicted in FIG. 4A and FIG. 4B. For example, the composite graft may be configured as a patch, such as graft 410 shown in FIG. 4A. The grafts may have a circular shape, a rectangular shape, an irregular shape, or some other shape, that is configured to fit the shape of the implant site 420. Such grafts may be relatively thin and flexible. In some instances, the composite graft may comprise a cylindrical shape as depicted in FIG. 4B and FIG. 4C. Such grafts may be configured as an osteochondral plug having a particular orientation, such as graft 440 in FIG. 4C. As discussed further elsewhere in this disclosure, composite grafts may include multiple distinct regions comprising different components that promote integration of the graft at the implantation site 420 and tissue growth, the positioning of the multiple distinct regions within the graft 440 imparting a particular orientation to the graft. In one example, the composite graft 440 shown in FIG. 4B and FIG. 4C comprises an osteogenic region and a chondrogenic region, which are discussed further elsewhere in this disclosure. Other cartilage and osteochondral graft shapes are also contemplated, such as, for example, graft shapes 440a-440k as shown in FIG. 4D. For example, graft shapes 440a, 440b, and 440f-440k each comprise possible osteochondral graft shapes. In another example, graft shapes 440c-e each comprise possible cartilage shapes.

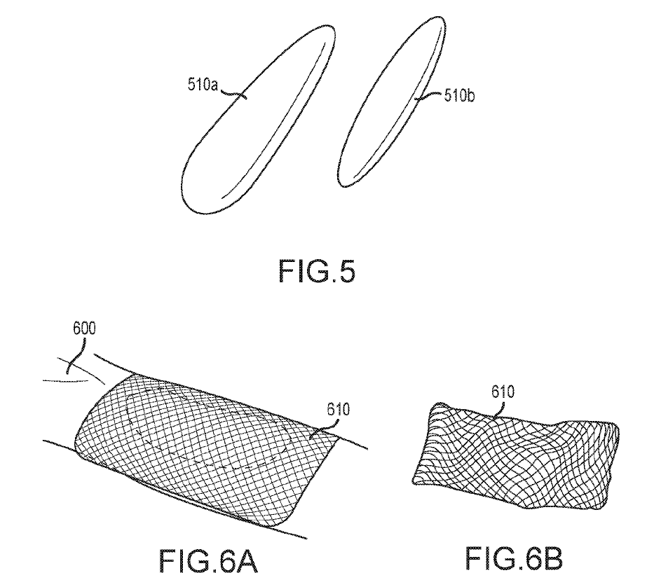

In some instances, the composite grafts may be configured in the shape of a muscle or portion thereof. Such grafts may have an irregular shape but will generally have an rounded exterior. A wide variety of shapes are contemplated for grafts configured in the shape of a muscle. Exemplary grafts 510a and 510b as shown in FIG. 5 may be oblong and oval in shape mimicking the shape of a long muscle (for example, as found in an arm or leg). In some instances, the grafts may be any of longer, shorter, narrower, wider, or more or less rounded than grafts 510a and 510b depicted in FIG. 5.

In some instances, the composite grafts may be configured as a sheet. An exemplary sheet graft 610 is shown in FIG. 6A and FIG. 6B. The grafts may be between 0.2 mm and 3 mm thick but may otherwise have various perimeter diameters and shapes. In some instances, the grafts may be continuous within their perimeter. In other instances, the grafts may be discontinuous such as the graft 610 shown in FIG. 6A and FIG. 6B. For example, the grafts may have a lattice-like, grid-like, or cross-hatched, configuration. Such grafts may be particularly useful for implantation on a body surface 600 of a subject to replace skin or facilitate skin growth as described elsewhere in this disclosure.

In some instances, the composite grafts may be fully or partially dehydrated. For example, if a composite graft does not include cells, the graft may be fully or partially dehydrated. In some instances, the grafts may be hydrated. Generally, grafts that contain cells will be at least partially hydrated. In some instances, the grafts may contain 0.5% water to 75% water content, in particular, may contain 10% to 40% water w/w. In some instances, the composite grafts may be stored in a biocompatible solution such as a cryopreservation medium or a nutritive media. For example, composite grafts, particularly those containing cells as a biological component, may be stored in a biocompatible medium. The nutritive medium may be a buffered solution or a growth medium. Exemplary buffered solutions include phosphate buffer saline, MOPS, HEPES, and sodium bicarbonate. The pH of the solution is generally in the range of pH 6.4 to 8.3. Suitable examples of growth medium include, but are not limited to, Dulbecco's Modified Eagle's Medium (DMEM) with 5% Fetal Bovine Serum (FBS). In some instances, growth medium may include high glucose DMEM. In some instances, the grafts may be stored at room temperature, refrigerated (approximately 5-8° C.), or frozen (approximately −20° C., −80° C., −120° C.). In some instances, the grafts may be cryopreserved such that the grafts include, or have been combined with or stored in, a cryopreservation medium. Cryopreservative medium may include one or more cryoprotective agents such as, but not limited to, glycerol, DMSO, hydroxyethyl starch, polyethylene glycol, propanediol, ethylene glycol, butanediol, or polyvinylpyrrolidone. In one example, a cryopreservation medium may include DMSO and glycerol. In some instances, the biocompatible solution may include an antibiotic.

A. Synthetic Scaffold

In one aspect, the grafts include a synthetic scaffold having a plurality of voids (empty spaces) defined therein. The scaffold comprises a trabecullar-like structure formed from an interconnected network of rod, beam, and/or strut projections with variability in the thickness and length of the projections. The rods, beams, and struts of the synthetic scaffold define the voids of the synthetic scaffold. The scaffold may be configured to have voids of varying shapes and sizes defined therein. In some instances, the entire scaffold structure may have a trabecular structure. In some instances, only a portion of the synthetic scaffold may be trabecular in nature. The voids defined in the synthetic scaffold may be on one or more surfaces of the scaffold, within one or more interior regions of the scaffold, or both. The configuration of the scaffold may be a regular lattice-like structure, an irregular lattice-like structure, or have one or more portions that are regular or irregular in structure. The scaffold is formed from a synthetic substrate. The three-dimensional shape of the scaffold may be based on the intended implantation site.

The configuration of the synthetic scaffold of the composite grafts may provide a three-dimensional space for tissue particles and cells. This configuration may permit ingrowth of native tissue from the defect site after implantation into a patient. In such instances, the synthetic scaffold component of the grafts may define at least one void configured to receive the native cells of the patient at the implantation site. The native tissue may be a bone tissue, cartilage tissue, epithelial tissue, muscle tissue, dermal tissue, or a combination thereof.

In some instances, the synthetic scaffold comprises at least one of an anatomical shape resembling a whole bone or a portion thereof comprising at least 10% of the whole bone and retaining at least some of the anatomical shape of the whole bone, a whole muscle or a portion thereof comprising at least 10% of the whole muscle and retaining at least some of the anatomical shape of the whole muscle, a portion of cartilage, or a portion of skin.

In one example, the synthetic scaffold may comprise an anatomical shape of a whole bone or a portion thereof comprising at least 10% of the whole bone. In another example, the synthetic substrate may comprise an anatomical shape of an anatomical shape of a whole muscle or a portion thereof comprising at least 10% of the whole muscle. For example, the synthetic substrate may comprise an anatomical shape of a portion of a whole bone or whole muscle comprising 10%, 15%, 20%, 25%, 30%, 35%, 40%, 45%, 50%, 55%, 60%, 65%, 70%, 75%, 80%, 85%, 90%, 95%, or 99% thereof. In some instances, where the synthetic scaffold comprises an anatomical shape of a portion of a whole bone or whole muscle, the portion may retain at least some of the anatomical shape of the whole bone or whole muscle.

In some instances, the synthetic scaffold has the anatomical shape of a portion of cartilage. As discussed elsewhere in this disclosure, cartilage may have a planar configuration. An example of a planar configuration is shown in FIG. 4A (showing graft 410 as a disc), however planar configurations may be in any shape (not just circular). Cartilage is also found elsewhere in the body in irregular anatomical shapes. In some instances, the synthetic scaffold may comprise an entire irregular anatomical shape of cartilage. In some instances, the synthetic scaffold may comprise an anatomical shape of a portion thereof comprising at least 10% of the entire irregular anatomical shape. Exemplary irregular cartilage shapes are shown in FIGS. 4B-4C. For example, the synthetic substrate may be a portion of an irregular anatomical shape of cartilage comprising 10%, 15%, 20%, 25%, 30%, 35%, 40%, 45%, 50%, 55%, 60%, 65%, 70%, 75%, 80%, 85%, 90%, 95%, or 99% thereof. In some instances, where the synthetic scaffold is a portion of an irregular anatomical shape of cartilage, the portion may retain at least some of the anatomical shape of the the irregular anatomical shape of cartilage.

In some instances, the synthetic scaffold has the anatomical shape of a portion of skin. As discussed elsewhere in this disclosure, skin has a planar configuration, generally in the form of a sheet. Exemplary configurations for synthetic scaffold having the anatomical shape of a portion of skin are shown in FIG. 6A and FIG. 6B (showing grafts 610), however the synthetic scaffold may have any 2-dimensional shape (not just rectangular).

In some instances, the synthetic scaffold may be in the shape of a cube, a strip, a sphere, or a wedge. In some instances, the synthetic scaffold is particulate in nature, meaning that is in the form of particles. In other instances, the synthetic scaffold is not particulate in nature, meaning that is not in the form of particles. The term particles and particulates refer to minute pieces of synthetic scaffold. The particles may be roughly spherical in shape and generally have a diameter of about 6 mm or less than and a volume less than 1 cm³. Particles may be roughly cubic or irregular in shape and generally have a height, width, and/or length of less than 10 mm and a volume less than 1 cm³. Exemplary particle sizes may include heights, widths, and/or lengths between about 0.1 mm and about 9 mm, between about 2 mm and about 8 mm, between about 1 mm and about 7 mm, between about 1 mm and about 6 mm, between about 1 mm and about 0.5 mm, between about 0.1 mm and about 4 mm, between about 1 mm and about 4 mm, or between about 0.1 rum and about 1 mm. Exemplary particle sizes may include a diameter between about 0.1 mm and about 6 mm, between about 0.1 mm and 1 mm, between about 1 mm and about 3 mm, between about 2 mm and about 5 mm, or between about 4 mm and about 6 mm.

In some instances, the synthetic scaffold may comprise a volume of 1 cm³ or greater. The synthetic scaffold may have a volume of at least 1 cm³, at least 1.5 cm³, at least 2 cm³, at least 2.5 cm³, or at least 3 cm³.

In some instances, the synthetic scaffold comprises a bioresorbable polymer. As used herein, bioresorbable indicates the quality of being able to be dissolved in the human body. For example, polyglycolic acid (a very common suture material), when implanted within the human body, is slowly hydrolytically broken down into water soluble glycolic acid salts that are later excreted from the body. Exemplary bioresorbable polymers include, but are not limited to, polylactides, polyglycolides, polyanhydrides, polycaprolactones, oxidized cellulose, alginate polymers or derivative thereof, fibrin polymers or derivatives thereof, or copolymers of any combination thereof. In some instances, the synthetic substrate may have been integrated with cellular adhesion molecules that support the physical attachment of cells. In some instances, the synthetic substrate may have structural integrity sufficient to maintain the physical properties of the composite graft and also be receptive to cellular proliferation and integration. The bioresorbable polymer may contain a single type of chemical monomer or multiple monomer types. Grafts having synthetic scaffolds comprising bioresorbable polymers may be useful for implantation at a defect site where they can provide solid support to the site after implantation and then be removed by physiological processes over time as native tissue grows into the defect site. In some instances, the bioresorbable polymer will have a melting temperature no greater than 50° C.

Depending on the intended use, different degrees of hardness/compressibility and flexibility may be desired for the composite graft. In one aspect, the hardness of the synthetic scaffold is a primary determinant of the overall strength and hardness of the composite grafts. The properties of the synthetic component, such as its configuration, degree of porosity, and chemical composition, may be selected to achieve a particular degree of hardness/compressibility, flexibility, or other adjustable quality in the graft. In some instances, where the intended implantation site for the composite graft is load bearing, the scaffold may be configured to have a high degree of hardness and little flexibility. In other instances, where the intended implantation site is soft tissue, the scaffold may be configured to have a high degree of compressibility, flexibility, or both.

The composite grafts of the disclosure may have various compressive strengths. As used herein, compressive strength means the capacity of a material or structure to withstand loads tending to reduce size. The compressive strength can be measured by plotting applied force against deformation in a testing machine. In some instances, composite grafts may be intended as a load-bearing implant. Examples of load-bearing implant sites can include, but are not limited to, degenerated or damaged spinal discs, long bone defects, cartilage defects, and osteochondral defects. In some instances, the composite grafts may be used for a non-load bearing implant site. Examples of non-load bearing implant sites can include, but are not limited to, oral or maxillofacial defects, cartilage defects, osteochondral defects, muscle defects, and skin defects. In some instances, load bearing implants will have greater compressive strengths than non-load bearing implants.

In some instances, osteogenic grafts may have a compressive strength in the range of 70 MPa to 1,400 MPa. For example, osteogenic grafts that mimic the strength of natural bone may have a compressive strength of 70-280 MPa. In one example, an osteogenic graft intended for replacement of cortical bone may have a compressive strength of 110-150 MPa. In one example, an osteogenic graft intended for replacement of cancellous bone may have a compressive strength of 2-6 MPa. In some instances, osteogenic grafts may have a compressive strength of 950-1,400 MPa (for example, when having a metal synthetic scaffold), which is significantly greater than the strength of natural bone. In some instances, chondrogenic implants may have a compressive strength in the range of 0.5 MPa to 15 MPa, which is similar to the compressive strength of natural cartilage. In some instances, vulnerary muscle implants may have a compressive strength in the range of 0.5 MPa to 20 MPa, which is similar to the compressive strength of natural muscle. In some instances, vulnerary skin implants may have a compressive strength in the range of 0.2 MPa to 7 MPa, which is similar to the compressive strength of natural skin. Table 1 below summarizes exemplary compressive strength ranges for different types of implants.

TABLE 1
Composite Graft Compressive Strengths

| | Compressive Strength |
|---|---|
| Graft Type | (Mega Pascal) |
| Osteogenic | 70-1,400 MPa |
| Cortical | 110-150 MPa |
| Cancellous | 2-6 MPa |
| Chondrogenic | 0.5-15 MPa |
| Vulnerary - muscle | 0.5-20 MPa |
| Vulnerary - skin | 0.2-7 MPa |

The composite grafts provided have one or more voids defined therein by the synthetic scaffold. The size of the voids in the grafts may be selected based on the dimensions of the biological component of the grafts. As the particle size of the biological component may vary, the voids defined in the graft may be similarly varied so as to accommodate the biological component. In some instances, the grafts may contain voids defined therein that have dimensions suitable for the ingrowth of native tissue after implantation. The grafts may contain voids of various different dimensions defined therein. Alternatively, the grafts may contain a set distribution of void sizes such that all voids defined therein have approximately the same dimensions or have dimensions within a specific range of dimensions. In some instances, the grafts may contain a random distribution of void sizes. In some instances, the grafts may contain voids of one or more specific ranges of dimensions defined therein or defined within specific regions thereof. In some instances, there may be a larger number of smaller voids defined in the grafts as compared to larger voids. In some instances, there may be a larger number of larger voids defined in the grafts as compared to smaller voids. For example, the majority of the voids defined in a graft may be relatively small and a minority of the voids may be relatively large and defined in the graft in a particular region of the graft or pattern therein. In another example, the majority of the voids defined in a graft may be relatively large and a minority of the voids may be relatively small and defined in the graft in a particular region of the graft or pattern therein. The voids defined in the grafts may be 10 μm-1 mm in diameter. In some instances, the voids may be 10 μm-75 μm in diameter. In some instances, the voids may be 75 μm-150 μm in diameter. In some instances, the voids may be 150-300 μm in diameter. In some instances, the voids may be 50 μm-100 μm in diameter. In some instances, the voids may be 100 μm-200 μm in diameter. In some instances, the voids defined in the grafts may be 100 μm-500 μm in diameter. In some instances, the voids may be 300 μm-500 μm in diameter. In some instances, the voids may be 500 μm-750 μm in diameter. In some instances, the voids may be 750 μm-1 mm in diameter.

In another aspect, the porosity of the synthetic scaffold of the composite grafts may range from 0% porous (non-porous) to up to 80% porous. For example, the porosity of the synthetic scaffold, in its entirety or a portion thereof, may be 1%, 2%, 5%, 10%, 15%, 20%, 25%, 30%, 35%, 40%, 45%, 50%, 55%, 60%, 65%, 75%, 80%, or a porosity within 2-3% of any of these percentages. In some instances, the location of the voids defined in the composite grafts may be the location of the biological component of the grafts. The porosity of the synthetic scaffold may be directly related to the amount of the biological component in the composite grafts. In some instances, the location of the voids defined in the composite grafts may be the location at which tissue ingrowth may occur after implantation at a defect site of a subject. In some instances, the graft may be uniformly porous such that voids are defined throughout the entirety of the synthetic scaffold. In some instances, the grafts may be nonporous or less porous in some portions of the scaffold, while other portions of the scaffold may contain voids or a relatively larger number of voids defined therein. In some instances, the synthetic scaffold of the grafts may have an internal portion that is nonporous and an external portion that is porous. In some instances, the synthetic scaffold of the grafts may be porous on one or more ends or one or more sides and nonporous in other areas or sides. In one example, a composite graft having the configuration of a long bone may have porosity at one end or both ends of the graft where it is intended to integrate into the implantation site by promoting tissue growth. In another example, a composite graft in the configuration of a sheet for use as a skin graft may have porosity only on the side of the graft to come into contact with the subject.

B. Biological Component

The composite grafts contain one or more biological component positioned in the voids of the scaffold. The biological component of the composite grafts may aid integration of the composite graft, regrowth of the native tissue, or both, after implantation of the graft at a defect site in a subject. The biological component may include one or more types of biological components including osteogenic biological components, chondrogenic biological components, and vulnerary biological components. As used herein, an osteogenic biological component refers to a biological component that promotes the growth or regrowth of bone tissue. As used herein, a chondrogenic biological component refers to a biological component that promotes the growth or regrowth of cartilage tissue. As used herein, a vulnerary biological component refers to a biological component that promotes the growth or regrowth of soft tissue such as skin or muscle, or healing thereof.

The biological component may include one or more of tissue particles, cells, or proteins (such as growth factors). Different types of biological components may be included in the composite grafts depending on the intended use of the grafts. As discussed, the grafts may contain one or more types of biological components including osteogenic biological components, chondrogenic biological components, and vulnerary biological components. For clarity, features of the biological components are first discussed generally, followed by a separate description of composite grafts containing different types of biological components.

The composite grafts may include a first biological component and a second biological component embedded in voids of the synthetic scaffold. In some instances, the first and second biological components are embedded in the synthetic scaffold during the additive manufacturing process in which the composite graft in synthesized. In some instances, the first biological component is embedded in the synthetic scaffold during the additive manufacturing process and the second biological component is embedded into voids of the synthetic scaffold using an agitation process. In some instances, the first and second biological components are the same type of biological component. In some instances, the first and second biological components are different types of biological components. In some instances, the first and second biological components are embedded uniformly throughout the voids of the synthetic scaffold. In some instances, the first biological component is embedded in voids of the scaffold having one shape and/or size, and the second biological component is embedded in voids of the scaffold having a different shape and/or size. In some instances, the first biological component is embedded in one portion of the synthetic scaffold and the second biological component is embedded in a different portion of the synthetic scaffold. For example, in some instances, the first biological component may be embedded or positioned in only some portions of the composite grafts such as in interior regions, and the second biological component may be embedded or positioned in voids defined in the surface of the scaffold or a portion thereof. In some instances, the grafts may include an equal proportion of the first biological component to the second biological component. In some instances, the grafts may include a greater proportion of the first biological component to that of the second biological component. In some instances, the grafts may include a greater proportion of the second biological component to that of the first biological component.

1. Configuration of Biological Component

In some instances, the biological component may include tissue particles. The tissue particles may be in the form of tissue particles, tissue strips, tissue ribbons, tissue shavings, or tissue in some other particulate form. The particles may be configured as circles, spheres, squares, rectangles, cubes, cylinders, strips, tiles (particles that are partially attached to other particles), or other desired shapes. The tissue particles may be generated by mincing, grinding, cryofracturing, or other known methods of generating particulate tissue. In some instances, the tissue particles are decellularized. For example, the tissue particles may be acellular or partially decellularized. In some instances, the tissue particles are not decellularized. Depending on the type of composite graft, the tissue particles may be osteogenic, chondrogenic, or vulnerary. For example, the tissue particles may be bone particles, cartilage tissue particles, muscle tissue particles, dermal tissue particles, or birth tissue particles. In some instances, the tissue particles may be collagen matrix derived from a tissue. Thus, in some instances, the biological component may include collagen matrix particles.

In some cases, the biological component may include cells. Depending on the type of composite graft, the cells may be osteogenic, chondrogenic, or vulnerary. For example, the cells may include mesenchymal stem cells, osteoblasts, chondrocytes, keratinocytes, platelet-rich plasma, or some combination of two or more thereof.

In some instances, the biological component may include tissue particles combined, or seeded, with cells. In some instances, the biological component may include tissue particles combined with growth factors.

The biological component may be obtained from a deceased donor, derived from deceased donor tissue, obtained from a living donor, or derived from living donor tissue. The biological component may be derived in whole or in part from a human donor. The biological component may be derived in whole or in part from a non-human animal such as, for example, non-human primates, rodents, canines, felines, equines, ovines, bovines, porcines, and the like. The biological component may be, or be derived from, an autograft tissue obtained from the intended recipient subject of the graft. The biological component may be, or be derived from, an allograft tissue obtained from an individual (donor) other than the intended recipient subject. In some instances, the biological component may be obtained or derived from a cadaveric donor such as a human cadaveric donor. Allograft tissue may be obtained from deceased donors that have donated their tissue for medical uses to treat living people. Such tissues represent a gift from the donor or the donor family to enhance the quality of life for other people. Allograft tissue may also be obtained as consented tissue from a living donor. Examples of consented tissue include dermal tissue, birth tissue, and adipose tissue. Donor tissue may be processed, transformed, or otherwise adjusted to provide the biological component.

The biological component may include tissue particles, alone or in combination with cells or proteins. The biological component particles may be of uniform size or may be various different sizes. For example, the particles may be uniform in size or have a size in a defined range. In some instances, the average diameter of tissue particles may be about 0.01 mm to about 5 mm. For example, the average diameter may be about 0.01 mm, about 0.02 mm, about 0.03 mm, about 0.04 mm, about 0.05 mm, about 0.06 mm, about 0.07 mm, about 0.08 mm, about 0.09 mm, about 0.1 mm, about 0.2 mm, about 0.3 mm, about 0.4 mm, about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.5 mm, about 3.0 mm, about 4.0 mm, about 4.5 mm, or about 5.0 mm. In some instances, the particles may have an average diameter of about 0.01 mm-5.0 mm, of about 0.05 mm to about 1.1 mm, of about 0.5 mm to about 5 mm, of about 0.05 mm to about 2.5 mm, of about 1 mm to about 5 mm, or of about 1 mm to about 3 mm. Such particle sizes may differ based on the tissue type of the deceased donor tissue. In some instances, the particles may be about 50 μm to about 1100 In some instances, the particles may be about 125 μm to about 1100 μm in average diameter.

In some instances, tissue particles and collagen matrix particles of a desired average diameter may be prepared using dual sieve apparatus. In one example, an upper sieve having 1100 μm diameter holes and a lower sieve having 50 μm diameter holes may be used. Particles that pass through the upper sieve and that are retained by the lower sieve can be considered to have a particle size within a range from 50 to 1100 μm. Other sized sieves may be used to isolate particles in different size ranges for use as the biological component. The collagen matrix particles may be particulates, fibres, or other shapes as described elsewhere herein.

The composite grafts may include biological components of a variety of sizes of tissue particles, cells, and proteins. Generally, the biological component is particulate in nature. The size of the biological component particle positioned within a void defined in scaffold may be proportional to the size of the void. In some instances, biological components having a smaller diameter may be embedded or positioned within smaller voids defined in the scaffold. In some instances, biological components having a larger diameter may be embedded or positioned within larger voids defined in the scaffold. By way of example, the biological component may be selected to be approximately the same size as at least a portion of the voids defined in the synthetic scaffold. In another example, the size of at least a portion of the voids defined in the synthetic scaffold may be selected to be approximately the same size as one of more of the biological components. In some instances, the biological component may be positioned tightly within at least a portion of the voids defined in the synthetic scaffold, wherein the tight fit facilitates retention of the biological component within the composite graft. Specifically, the biological component may be held into place within the voids as a result of friction present between the biological component and the scaffold (synthetic or bone). In being frictionally held into place within a void of the scaffold, a biological component particle is restrained from motion by frictional force; that is frictionally held in place by the scaffold defining the void. As shown in FIG. 1B and FIG. 1C, the voids defined in the scaffold act like pockets into which biological components may be positioned and restrained. In some instances, the biological component may be positioned or embedded in the voids defined in the synthetic scaffold such that the biological component protrudes from the voids. In some instances, the voids may be defined in the surface of the synthetic scaffold and the biological component may protrude from the surface of the scaffold itself. In some instances, a portion of the biological component within the scaffold may be held within the voids by friction. In some instances, all of the biological component within the scaffold may be held within the voids by friction.

In some instances, the biological component may be embedded or positioned uniformly amongst the voids of the synthetic scaffold such that there is a relatively uniform distribution of the biological component amongst the voids or within different portions of the grafts. In some instances, the biological component may be embedded or positioned non-uniformly throughout the voids of the scaffold such that some portions of the grafts may include a greater proportion of biological component that other portions of the grafts. For example, in some instances, the biological component may be embedded or positioned in only some portions of the composite grafts such as along one or more sides or in one or more regions. In some instances, the biological component may be embedded or positioned in only voids defined in the surface of the scaffold or a portion thereof.

The voids of the composite grafts may be saturated to various degrees with the biological component. In some instances, a majority of the voids defined in the scaffold have a biological component positioned therein. In some instances, a minority of the voids defined in the scaffold have a biological component positioned therein. In some instances, almost all of the voids defined in the scaffold have a biological component positioned therein. The percent saturation of the voids defined in the scaffold with biological component may range from 1% to 100%. For example, the percent saturation may be 1%, 2%, 5%, 10%, 15%, 20%, 25%, 30%, 35%, 40%, 45%, 50%, 55%, 60%, 65%, 75%, 80%, 85%, 90%, 95%, 100%, or a porosity within 2-3% of any of these percentages. Different portions of the composite grafts may be saturated to different degrees. For example, some portions of the grafts may contain biological component positioned or embedded within at least a portion of the voids defined therein. In another example, one or more portions of the composite grafts may not contain any biological component.

2. Osteogenic Grafts

In some instances, the composite grafts provided are osteogenic grafts. The biological components of the composite grafts may include one or more osteogenic biological components. Osteogenic biological components may promote bone growth in vivo at a defect site. Osteogenic components may be osteoinductive, osteoconductive, or both. Osteoinductive bone formation involves the formation of new bone by the attraction of osteoblasts. Osteoconductive bone formation involves a slower process of providing a structure/scaffold to promote new bone growth. Composite grafts containing osteogenic biological components are generally useful to treat bone defects. Osteogenic biological components may include one or more of osteogenic tissue particles, osteogenic cells, and osteogenic growth factors. The osteogenic tissue particles may include at least one of bone particles or acellellular collagen matrix particles. The osteogenic cells may include at least one of mesenchymal stem cells, osteoblasts, or platelet-rich plasma (PRP).

Osteogenic grafts may be useful in a variety of indications including, for example, neurosurgical and orthopedic spine procedures. In some instances, osteogenic grafts can be used for purposes such as fusing joints or adjacent bones, repairing broken bones, and replacing missing bones or portions of bones.

In some instances, the osteogenic tissue particles may include bone particles. The bone particles may be mineralized bone, demineralized bone, or a combination thereof. The bone particles may be fully demineralized, partially demineralized, or fully mineralized. The American Association of Tissue Banks typically defines demineralized bone matrix as containing no more than 8% residual calcium as determined by standard methods. In this sense, fully demineralized bone can be considered to have no more than 8% residual calcium. The bone particles may be cancellous bone, cortical bone, or combinations thereof. In some instances, the bone particles may be demineralized bone matrix (DBM). DBM refers to bone that has had inorganic mineral removed, leaving behind the organic collagen matrix. The bone particles may be in various forms including bone particles, bone strips, bone ribbons, and bone shavings, or a combination thereof. In some instances, the bone particles may be ground, minced, morselized, or otherwise particulated bone.

In some instances, the osteogenic tissue particles may include particles of acellular collagen matrix. In some cases, the acellular collagen matrix may comprise primarily type I collagen. For example, the acellular collagen matrix may be acellular dermal collagen matrix. The collagen matrix may be particulate in form such as, for example, in the form of particles, strips, ribbons, and shavings, or a combination thereof. In some instances, the collagen matrix may be ground, minced, morselized, or otherwise particulated collagen matrix.

In some instances, the osteogenic tissue particles may include particles of acellular collagen matrix. In some cases, the acellular collagen matrix may comprise primarily type I collagen. For example, the acellular collagen matrix may be acellular dermal collagen matrix. Decellularization of the collagen matrix may reduce immunogenicity of the composite grafts. The collagen matrix may be particulate in form such as, for example, in the form of particles, strips, ribbons, and shavings, or a combination thereof. In some instances, the collagen matrix may be ground, minced, morselized, or otherwise particulated collagen matrix.

The osteogenic biological component may include osteogenic cells or a cell-containing component. In some instances, the osteogenic cells or a cell-containing component may be one or more of mesenchymal stem cells, osteoblasts, and platlet-rich plasma.

In some instances, the osteogenic cells may include mesenchymal stem cells. Mesenchymal stem cells (MSC) are multipotent stromal cells that can differentiate into a variety of cell types, including osteoblasts, chondrocytes, myocytes and adipocytes. The mesenchymal stem cells may be derived from any of a number of different tissues including, but not limited to adipose tissue, muscle tissue, birth tissue (such as amnion or amniotic fluid), skin tissue, bone tissue, or bone marrow tissue. The mesenchymal stem cells may be cultured in vitro prior to inclusion in the composite grafts such as for the purposes of proliferating and/or enriching the mesenchymal stem cells. Alternatively, the mesenchymal stem cells may not be cultured in vitro prior to inclusion in the composite grafts such that the cells may be isolated and then used directly in the manufacture of the grafts. For example, in some instances, the mesenchymal stem cells may used as the biological component in the composite grafts without prior proliferation or enrichment by in vitro culturing (such as on tissue culture plastic).

In some instances, the osteogenic cells may include osteoblasts or osteoblast-like cells. Osteoblasts are cells that secrete an extracellular matrix and direct its subsequent mineralization to form bone. Osteoblasts may be isolated from bone tissue. In some instances, the osteoblasts are cultured in vitro (such as in an explant culture) prior to inclusion in the composite grafts. In some instances, the osteoblasts are not cultured in vitro prior to inclusion in the composite grafts. As used herein, osteoblast-like cells include osteoblast precursor cells or cells that will behave like osteoblasts when in an environment that promotes osteogenesis (such as one having bone morphogenic proteins present). In some instances, the trabecular/porous nature of the synthetic scaffold of the composite grafts may promote retention of osteoblasts and osteoblast-like cells within the scaffold, promote viability of cells within the scaffold, or both.

In some instances, the osteogenic cells include platelet-rich plasma (PRP), which is blood plasma that has been enriched with platelets. PRP contains (and releases through degranulation) several different growth factors and other cytokines that stimulate healing of bone and soft tissue.

In some instances, the osteogenic biological component may include a combination of tissue particles and cells. For example, the osteogenic biological component may include bone particles combined or seeded with mesenchymal stem cells. In another example, the osteogenic biological component may include particles of acellular collagen matrix, such as type I collagen matrix, combined or seeded with mesenchymal stem cells. Either or both of the bone tissue and collagen matrix may be particulate in form such as, for example, in the form of particles, strips, ribbons, and shavings, or a combination thereof. In some instances, the bone tissue and/or collagen matrix may be ground, minced, morselized, or otherwise particulated. Exemplary stem cell-seeded bone tissue and collagen matrix particles and methods of preparing such seeded particles are described in U.S. Pat. No. 9,192,695 and U.S. Patent Application Publication No. 2014/0286911, the contents of each of which are incorporated by reference herein. In another example, the osteogenic biological component may include birth tissue particles combined or seeded with mesenchymal stem cells. Birth tissue as used herein refers to amniotic sac (including the amnion and chorion layers either together in their natural configuration or either separately), placenta, umbilical cord, and cells from fluid contained in each. Any of these tissues may be processed into particles (as described above) and combined with mesenchymal stem cells. The birth tissue particles may act as a stable carrier for the stem cells. In some instances, the birth tissue is amnion tissue or placental tissue, or a combination thereof. The birth tissue may be particulate in form such as, for example, in the form of particles, strips, ribbons, and shavings, or a combination thereof. In some instances, the birth tissue may be ground, minced, morselized, or otherwise particulated birth tissue.

The osteogenic biological component may include osteogenic growth factors such as bone morphogenic proteins (BMPs). BMPs are growth factors that induce the formation of bone. BMPs may be isolated from bone tissue or may be recombinant. Exemplary BMPs include, but are not limited to, BMP1, BMP2, BMP3, BMP4, BMP5, BMP6, BMP8a, BMP8b, BMP10, BMP15. In some instances, the biological component may contain one or more bone morphogenic proteins combined with a acellular collagen matrix tissue particles as a carrier. Commercial examples of such combinations include INFUSE® Bone Graft containing BMP2 (Medtronic, Minneapolis, Minn.) and Osteogenic Protein 1 (OP-1) Implant containing BMP7 (Stryker, Kalamazoo, Mich.).

3. Chondrogenic Grafts

In some instances, the composite grafts provided are chondrogenic grafts. The biological component may include one or more chondrogenic biological components. Chondrogenic biological components may promote cartilage growth in vivo at a defect site. Composite grafts containing chondrogenic biological components are generally useful to treat cartilage defects. Chondrogenic biological components may include one or more of chondrogenic tissue particles, chondrogenic cells, and chondrogenic growth factors. The chondrogenic tissue particles may include at least one of cartilage tissue particles or acellellular collagen matrix particles. The chondrogenic cells may include at least one of mesenchymal stem cells, chondrocytes, or platelet-rich plasma (PRP).

In some instances, the chondrogenic tissue particles may include cartilage tissue particles. Cartilage is generally flexible but inelastic cords of strong fibrous collagen-containing tissue that cushions bones at joints and makes up other parts of the body. Articular cartilage provides a smooth, lubricated surface for articulation and facilitates the transmission of loads with a low frictional coefficient. Chondrocytes generate proteins (for example, collagen, proteoglycan, and elastin) that are involved in the formation and maintenance of the cartilage. For example, articular cartilage contains significant amounts of collagen. Cross-linking of the collagen fibers may impart a high material strength and firmness to the cartilage tissue. The cartilage tissue particles may be partially decellularized or not decellularized. In some instances, the cartilage particles may include native chondrocytes. The cartilage tissue particles may be in various forms including cartilage particles, cartilage strips, cartilage ribbons, and cartilage shavings, or a combination thereof. In some instances, the cartilage tissue particles may be ground, minced, morselized, or otherwise particulated cartilage. In some instances, the cartilage tissue may include the cartilage tissue described in U.S. Patent Publication No. 2014/0134212, filed Nov. 15, 2013, U.S. Patent Publication No. 2014/0243993, filed Feb. 21, 2014, and U.S. Patent Publication No. 2014/0271570, filed Mar. 13, 2014, the entire contents of each of which are incorporated herein by reference.

In some instances, the chondrogenic tissue particles may include particles of acellular collagen matrix. In some cases, the acellular collagen matrix may comprise primarily type II collagen. Cross-linking of the collagen fibers may impart a high material strength and firmness to the collagen matrix. For example, the acellular collagen matrix may be acellular cartilage collagen matrix. Decellularization of the collagen matrix may reduce immunogenicity of the composite grafts. The collagen matrix may be particulate in form such as, for example, in the form of particles, strips, ribbons, and shavings, or a combination thereof. In some instances, the collagen matrix may be ground, minced, morselized, or otherwise particulated collagen matrix.

The chondrogenic biological component may include chondrogenic cells or a cell-containing component. In some instances, the chondrogenic cells or a cell-containing component may be one or more of mesenchymal stem cells, chondrocytes, and platelet-rich plasma (PRP). The discussion above with respect to MSC and PRP is applicable here as well. Chondrocytes are the only cells found in native cartilage. Chondrocytes produce and maintain the cartilaginous matrix, which consists mainly of collagen and proteoglycans.

In some instances, the chondrogenic biological component may include a combination of tissue particles and cells. The biological component may contain cartilage tissue particles combined or seeded with mesenchymal stem cells. The biological component may contain cartilage tissue particles combined or seeded with chondrocytes. The biological component may contain acellular type II collagen matrix combined or seeded with mesenchymal stem cells. The biological component may contain acellular type II collagen matrix combined or seeded with chondrocytes. Exemplary stem cell-seeded cartilage tissue and collagen matrix particles and methods of preparing such seeded particles are described in U.S. Patent Application Publication Nos. 2014/0024115 and 2014/0286911, the contents of each of which are incorporated by reference herein.

The chondrogenic biological component may include chondrogenic growth factors. As used herein, chondrogenic growth factors are growth factors also known as cytokines and metabologens which can induce the formation of cartilage (chondrogenic). In some instances, the biological component may contain one or more chondrogenic growth factors combined with a acellular collagen matrix tissue particles as a carrier. Chondrogenic growth factors can be isolated from tissue or recombinant.

Chondrogenic grafts may be useful in a variety of ways to treat cartilage defects. For example, articular cartilage is not vascularized, and when damaged as a result of trauma or degenerative causes, has little or no capacity for in vivo self-repair. The composite grafts provided may aid healing by delivering reparative cells or tissues. For example, when grafts containing cartilage particles are implanted into a patient at a cartilage defect site, chondrocytes may migrate out of the grafts and carry out repair and regeneration functions. For example, the chondrocytes can reproduce and form new cartilage via chondrogenesis. In this way, a composite graft containing cartilage can be applied to a site within a patient to treat cartilage defects. For example, chondrocytes from the grafts can reproduce and generate new cartilage in situ. The newly established chondrocyte population and cartilage tissue can fill defects and integrate with existing native cartilage and/or subchondral bone at the treatment site. Grafts containing mesenchymal stem cells may similarly heal cartilage defects as the cells may differentiate into chondrocytes. Grafts containing growth factors may facilitate healing of cartilage defects by stimulating chondrogenesis in native chondrocytes present at the implantation site.

4. Osteochondral Grafts

In some instances, the composite grafts provided are osteochondral grafts. The biological component may include an osteogenic component, a chondrogenic component, or a combination thereof, as described above. Osteogenic biological components may promote bone growth in vivo at a defect site. Chondrogenic biological components may promote cartilage growth in vivo at a defect site. Composite grafts containing biological components that are osteogenic, chondrogenic, or both, are generally useful to treat osteochondral defects. An osteochondral defect is an injury to the smooth surface on the end of bones, called articular cartilage (chondro), and the bone (osteo) underneath it. The degree of injury ranges from a small crack to a piece of the bone breaking off inside the joint. Such defects also include a tear or fracture in the cartilage covering one of the bones in a joint. The cartilage can be torn, crushed or damaged and, in rare cases, a cyst can form in the cartilage. Osteochondral defects are common in the knee and ankle joints but may occur in other joints as well.

As discussed above, the osteogenic biological components may include one or more of osteogenic tissue particles, osteogenic cells, and osteogenic growth factors. The osteogenic tissue particles may include at least one of bone particles or acellular collagen matrix particles. The osteogenic cells may include at least one of mesenchymal stem cells, osteoblasts, or platelet-rich plasma (PRP). Also as discussed above, the chondrogenic biological components may include one or more of chondrogenic tissue particles, chondrogenic cells, and chondrogenic growth factors. The chondrogenic tissue particles may include at least one of cartilage tissue particles or acellular collagen matrix particles. The chondrogenic cells may include at least one of mesenchymal stem cells, chondrocytes, or PRP.

A particular feature of osteochondral grafts may be that different types of biological components may be positioned in distinct portions of the grafts. For example, osteochondral grafts may have a bone-facing, or bone-contacting, portion, and a cartilage-facing, or cartilage-contacting portion. As discussed above, exemplary osteochondral grafts are shown in FIG. 4B and FIG. 4C. In some instances, the bone-contacting portion of the grafts may have an osteogenic biological component positioned within voids defined therein. In some instances, the cartilage-contacting portion of the grafts may have an chondrogenic biological component positioned within voids defined therein.

In some instances, the biological component of the composite grafts is both osteogenic and chondrogenic. For example, the biological component may be at least one of mesenchymal stem cells or platelet-rich plasma. Each of these components promote both osteogenesis and chondrogenesis.

In some instances, as discussed above, the composite grafts may include voids defined therein only in specific regions or portions. For example, composite grafts may be porous on a bone-contacting portion of the grafts. In another example, composite grafts may be porous on a cartilage-contacting portion of the grafts. Grafts having such configurations may comprise either an osteogenic biological component or a chondrogenic biological component, respectively, wherein the biological component is positioned within the voids defined in the grafts. In one example, composite grafts may have a cylindrical configuration with voids defined in one end of the cylinder, and a biological component comprising minced cartilage tissue particles positioned within the voids. Such grafts may be used in a manner similar to that described in U.S. Pat. No. 8,702,809, wherein the porous region is implanted into a an osteochondral defect in a knee or other joint to promote the regeneration of hyaline cartilage in the defect. In another example, composite grafts may have a plug configuration as described in U.S. Pat. No. 9,168,140, with voids defined in cartilage-contacting portion (such an upper cap or dome region) adjacent to a nonporous bone-contacting portion (such as a lower stem or plug region), wherein a biological component comprising minced cartilage tissue particles is positioned within the voids. In either of these examples, the biological component may be any of the osteogenic biological components described in this disclosure.

5. Vulnerary Grafts

In some instances, the composite grafts provided are vulnerary grafts. The biological component may include one or more vulnerary component. Vulnerary biological components may promote soft tissue growth, or healing of soft tissue, in vivo at a defect site. Composite grafts containing vulnerary biological components are generally useful to treat soft tissue defects. Different types of vulnerary biological components may promote growth and/or healing of different types of soft tissue. For example, some vulnerary components may promote growth and/or healing of muscle tissue. In another example, some vulnerary components may promote growth and/or healing of skin tissue. In another example, the vulnerary components may promote growth and/or healing of soft tissue generally. The vulnerary biological component may include one or more of tissue particles or cells. The tissue particles, the cells, or both may be derived or obtained from a soft tissue. The soft tissue used as the source of the vulnerary component may be of the same type as at the intended implantation site for the composite grafts. Exemplary tissue particles include those described in U.S. Pat. No. 9,162,011, the entire content of which is incorporated by reference herein.

Vulnerary grafts suitable for implantation at a muscle defect may be referred to as muscle composite grafts. The vulnerary component of muscle composite grafts may include one or more of tissue particles or cells that promote muscle tissue growth and/or healing. The tissue particles may be muscle tissue particles or acellular collagen matrix derived from muscle tissue. The tissue particles or collagen matrix may be in the form of particles, strips, ribbons, shavings, or some other particulate form. The tissue particles may be partially decellarized or not decellularized. In some instances, muscle composite grafts may include mesenchymal stem cells or platelet-rich plasma (PRP) as the vulnerary component. In some instances, the biological component of muscle composite grafts may include mesenchymal stem cells, PRP, or both, combined with, or seeded on, muscle tissue particles or acellular collagen matrix particles derived from muscle tissue. Exemplary stem cell-seeded collagen matrix and methods of preparing such are described in U.S. Patent Application Publication No. 2014/0286911, the content of which is incorporated by reference herein.

Vulnerary grafts suitable for implantation at a skin defect may be referred to as dermal composite grafts. The vulnerary component of dermal composite grafts may include one or more of tissue particles or cells that promote skin tissue growth and/or healing. The tissue particles may be dermal tissue particles or acellular collagen matrix derived from dermal tissue. The tissue particles or collagen matrix may be in the form of particles, strips, ribbons, shavings, or some other particulate form. The tissue particles may be partially decellularized or not decellularized. In some instances, dermal composite grafts may include mesenchymal stem cells or keratinocytes. In some instances, the biological component of dermal composite grafts may include mesenchymal stem cells, keratinocytes, or both, combined with, or seeded on, dermal tissue particles or acellular collagen matrix particles derived from dermal tissue. In some instances, dermal composite grafts may include dermal tissue particles as the vulnerary component. For example, the dermal tissue particles may be partial thickness skin tissue particles. Grafts having partial thickness skin tissue particles as the biological component may lead to an immune response that facilitates sloughing off of the graft as skin tissue regrows at the defect site at the site of implantation.

C. Biological Adhesive

In some instances, the composite grafts may include a biological adhesive. A biological adhesive may strengthen the interaction between the synthetic scaffold and the biological component. In some instances, the biological adhesive may be used to facilitate adherence of tissue particles, including collagen matrix particles, within the voids defined in the synthetic scaffold. A biological adhesive may be particularly useful to facilitate adherence of smooth tissue particles that are relatively slippery or slick, such as minced cartilage. The biological adhesive may be used to facilitate adherence of cells to the synthetic scaffold. In some instances, the biological adhesive may be used to facilitate adherence of growth factor containing particles to the synthetic scaffold. The biological adhesive may be in the form of a putty or a paste. Suitable biological adhesives include, but are not limited to, fibrin, fibrinogen, thrombin, fibrin glue (such as, for example, TISSEEL), polysaccharide gel, cyanoacrylate glue, gelatin-resorcin-formalin adhesive, collagen gel, synthetic acrylate-based adhesive, cellulose-based adhesive, basement membrane matrix (such as, for example, MATRIGEL® (BD Biosciences, San Jose, Calif.)), autologous glue, carboxymethyl cellulose, laminin, elastin, proteoglycans, and combinations thereof. The amount of biological adhesive used may be the minimum amount to achieve the desired effect, of facilitating the adherence of the biological component to the synthetic scaffold.

### III. Methods of Treatment

The composite grafts provided are useful for treating a tissue defect in a subject (also referred to herein as a patient). As used herein, a tissue defect refers to a biological tissue that is damaged or diseased due to injury, disease, or iatrogenic processes. Use of the grafts may be implemented in industries related to orthopedics, reconstructive surgery, podiatry, and cartilage replacement. In some instances, the composite grafts provided may be reabsorbed and replaced with the patient's natural tissue upon healing. In some instances, the composite grafts are retained long term in a subject after implantation, replacing the missing or damaged tissue. The composite grafts may also have reconstructive applications, for example, in the context of missing sections of tissue or bone (such as from a wound). In some instances, the composite grafts of this disclosure provide tailored treatment options in terms of shape, size, and composition for treating a wide array of tissue defects. In some instances, the composite grafts may be used for post-traumatic reconstructive cosmetic uses. The treatment methods are generally performed by a medical professional such as a surgeon.

Provided are methods of treating a tissue defect in a subject, wherein treatment includes administering to the subject a composite graft at a defect site (also referred to herein as implantation site) in the subject. The defect site is a tissue defect site such as a degenerated or damaged spinal disc, a bone defect, an oral defect, a maxillofacial defect, a cartilage defect, an osteochondral defect, a muscle defect, or a skin defect. The subject may be a human or a non-human animal such as, for example, a non-human primate, a rodent, a dog, a cat, a horse, a pig, a cow, a bird, and the like. In some instances, the subject is a human.

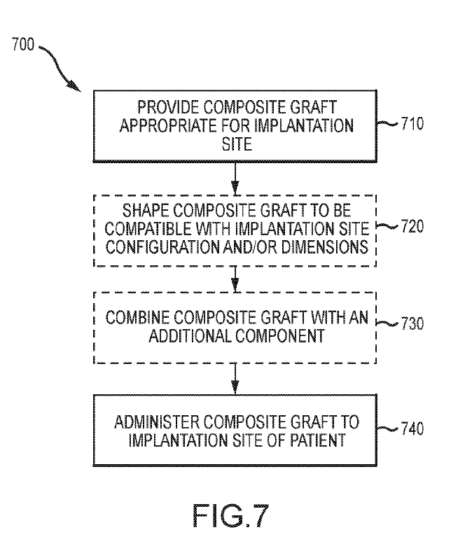

In some instances, an exemplary method of treatment 700 is shown as flow chart in FIG. 7. The method includes step 710 of providing a composite graft appropriate for the implantation site. This step may be performed following an evaluation of the patient. The medical professional evaluates a subject to determine the nature of the tissue defect that requires treatment and the type of composite graft appropriate to treat the subject. In some instances, this process may include medical imaging, such as any of X-ray imaging, MRI scans, or CT scans, which provide dimensions of the defect site, and may be utilized for determining the desired configuration (such as size, shape) of the graft. The appropriate composite graft may have a biological component selected to promote tissue growth and healing at the defect site. For example, an osteogenic composite graft may be appropriate to treat a bone defect. In another example, a chondrogenic composite graft may be appropriate to treat a cartilage defect. In another example, a osteochondrogenic graft may be appropriate to treat an osteochondral defect. In another example, a vulnerary graft may be appropriate to treat a soft tissue defect. In some instances, the biological component may be derived from tissue similar to the native tissue type at the defect site of the patient. For example, for a defect site that is a bone defect site, the biological component of the composite graft may be bone or bone-derived. In another example, the biological component may be muscle tissue, or derived therefrom, where the defect site includes a muscle defect. In some instances, the appropriate composite graft may include a biological component that is a different type of tissue, or derived from a different type of tissue, than is native of the defect site. For example, in some instances, an appropriate composite graft for treating a bone defect or an osteochondral defect may include birth tissue particles (such as birth tissue particles combined with mesenchymal stem cells or osteoblasts).

In some instances, shown as step 720, the composite graft may be shaped by the medical professional to be compatible with the configuration and/or dimensions of the implantation site. It is contemplated that the implant may be shaped such as by cutting, bending, folding, and the like. For example, the composite graft may be trimmed with a surgical tool, such as a scapel or scissors, to fit into a defect site. In some instances, this step may include hydrating or rehydrating a composite graft that is at least partially dehydrated. In some instances, the graft may be washed or rinsed to remove debris or solution in which the graft was stored.

In some instances, shown as step 730, the composite graft may be contacted or combined with an additional component prior to administration. Exemplary additional components include physiological saline, an antibiotic, autologous blood, platelet-rich plasma, or a combination of any thereof.

The composite graft is administered to the implantation site of the subject, which is shown as setup 740. The graft may be implanted into, or within, a defect site. For example, an osteogenic graft may be implanted into a defect site in which the native bone is missing (whether through damage, disease, or surgical removal). Chondrogenic, osteochondrogenic, and vulnerary grafts for treating cartilage, osteochondral, and muscle defects may be similarly implanted within a defect site. In some instances, composite grafts may be implanted, or placed, onto a defect site. For example, a vulnerary graft for treating a skin defect may be placed onto a defect site (for example, a burn site) on the surface of a patient's body. In some instances, a biological adhesive may be used to fix the composite graft into place at the implantation site. In some instances, the composite graft may be sutured or afixed with fasteners (such as screws) at the implantation site. For example, a vulnerary graft for treating a skin defect may be sutured or adhered to the implantation site. In another example, an osteogenic graft may be adhered, affixed with fasteners, or both into the implantation site.

In some instances, the tissue defect and, thus, the implantation site (also referred to as an implant site) may be a bone defect, a cartilage defect, an osteochondral defect, a skin defect, and/or a muscle defect. In some instances, the tissue defect/implant site may include a void in the subject's body defining the location of a removed portion of tissue. For example, the tissue defect/implant site may a location previously occupied by a tumor, such as a breast or bone tissue tumors, or a site related to reconstructive surgery applications such as, for example, wound sites or sites where native tissue has degraded. For example, the composite grafts may implanted into a defect site to act as a cartilage replacement to maintain a structural shape (such as for nose reconstruction, ear configurations) or function (such as for ACL replacement), a bone replacement (such as for ribcage reconstruction, long bone reconstruction, or spinal disc replacement), a muscle tissue replacement (such as for muscle reconstruction), or a skin replacement (such as for a burn wound).

The methods provided may include administering a composite graft to treat a subject having a bone defect. Exemplary bone defects include damaged, diseased, degenerated, or missing bones. For example, the defect site may be a long bone, a short bone, a flat bone, an irregular bone, an intervertebral disc, or a portion of any of these bones. In some instances, the bone defect may be an oral defect, a maxillofacial defect, or a combination thereof. In some instances, the bone defect may be a joint defect. In some instances, the bone defect may be a damaged or diseased intervertebral disc. The methods may include administering an osteogenic composite graft to a patient with a bone defect, the osteogenic composite graft containing an osteogenic biological component. In some instances, the composite graft may facilitate bone repair, promote bone growth, and/or or promote bone regeneration at the defect site/implant site in the subject. In some instances, osteogenic biological components such as mesenchymal stem cells or osteoblasts can migrate out of the implanted graft and carry out repair and regeneration functions. For example, the osteoblasts can reproduce and form new bone via osteogenesis. The newly established osteoblast population can fill defects and integrate with existing native bone at the implantation site. In this way, osteogenic composite grafts that are implanted at a defect site within a patient may treat bone defects. In some instances, the grafts are selected, or are shaped, to mimic the configuration of the bone defect. In some instances, the osteogenic composite grafts may be bioresorbable by virtue of the bioresorbable synthetic scaffold. Such grafts may be absorbed by the subject's body over time as the osteogenic biological component facilitates healing of the bone defect.

In some instances, tissue defect/implant site may be a damaged or diseased long bone. For example, the tissue defect/implant site may be a site where cancerous bone has been removed. In another example, the tissue defect/implant site may be a traumatic wound site containing damaged or missing bone (such as from an accident or military wound). The grafts may be administered to a subject to repair a missing or damaged long bone or to promote bone growth or regeneration in the subject. In some instances, the subject may have a degenerative defect or injury. In some instances, the subject may have a traumatic defect or injury. In some instances, the composite graft may be implanted to replace an entire long bone or a portion thereof. Exemplary grafts for use to treat such defects are shown, or readily apparent from, FIG. 2F and FIG. 2I.

In some embodiments, the method may include administering an implant to a patient with an oral defect, a maxillofacial defect, or a combination thereof. As used herein, oral and maxillofacial defects include defects in the head, neck, face, jaws, and the hard and soft tissues of the oral (mouth) and maxillofacial (jaws and face) region. In some instances, the subject may have a degenerative defect or injury. In some instances, the subject may have a traumatic defect or injury. In some instances, the methods are for treatment (repair) of tooth defects, such as degenerated, broken, or missing teeth and, in some instances, degenerated, broken, or missing bone underlying such teeth. In some instances, the methods are for treatment (repair or reconstruction) of degenerated, broken, or missing bone from the head, neck, face, and/or jaws. Exemplary grafts for use to treat such defects are shown, or readily apparent from, FIGS. 2B-2E.

In some instances, tissue defect/implant site may be a damaged or diseased intervertebral disc. The method may include administration of the implant to a patient after a damaged or diseased intervertebral disc has been surgically removed. The method of administration may be referred to as spinal arthrodesis or spinal fusion. The biological component in the composite grafts may be an osteogenic biological component that promotes bone growth. As osteogenesis occurs at the implantation site, the intervertebral discs flanking the implanted composite graft may fuse to the graft, thereby stabilizing the spine. Exemplary grafts for use to treat such defects are shown, or readily apparent from, FIG. 2F and FIG. 2I. The implant may be selected such that the surface area of an upper and lower contact surfaces of the implant, and the height of the implant, are similar to the IVD surface area and height of the intervertebral disc being replaced with the implant.

The methods provided may include administering a composite graft to treat a subject having a cartilage defect. Exemplary cartilage defects include damaged, diseased, degenerated, or missing cartilage, ligament, tendon, or meniscus. In some instances, the bone defect may be a nasal cartilage defect, an ear cartilage defect, or a joint cartilage defect. In some instances, the cartilage defect may be a degenerative defect or injury. In some instances, the cartilage defect may be a traumatic defect or injury. In some instances, the cartilage defect may be osteoarthritis. The methods may include administering a chondrogenic composite graft to a patient with a cartilage defect, the chondrogenic composite graft containing a chondrogenic biological component. In some instances, the composite graft may facilitate cartilage repair, promote cartilage growth, and/or or promote cartilage regeneration at the defect site/implant site in the subject. In some instances, chondrogenic biological components such as mesenchymal stem cells or chondrocytes can migrate out of the implanted graft and carry out repair and regeneration functions. For example, the chondrocytes can reproduce and form new cartilage via chondrogenesis. The newly established chondrocyte population can fill defects and integrate with existing native cartilage and/or subchondral bone at the implantation site. In this way, chondrogenic composite grafts that are implanted at a defect site within a patient may treat cartilage defects. Exemplary grafts for use to treat such defects are shown, or readily apparent from, FIG. 3A (nasal defects), FIG. 3B (ear defects), and FIG. 4A (joint defect such as knee defect). In some instances, the grafts are selected, or are shaped, to mimic the configuration of the cartilage defect. In some instances, the chondrogenic composite grafts may be bioresobable by virtue of the bioresorbable synthetic scaffold. Such grafts may be absorbed by the subject's body over time as the vulnerary biological component facilitates healing of the cartilage defect.

In some embodiments, the methods provided may include administering a composite graft to treat a subject having an osteochondral defect. As used herein, an osteochondral defect refers to a focal area with cartilage damage and injury of the adjacent/underlying subchondral bone. One example of an osteochondral defect is osteochondritis dissecans, which may be used synonymously with osteochondral injury or osteochondral defect in the pediatric population. The methods may include administering an osteochondral composite graft to a patient with an osteochondral defect, the chondrogenic composite graft containing at least one of an osteogenic biological component or a chondrogenic biological component. As described above with respect to osteogenic grafts and chondrogenic grafts, the biological components of osteochondral composite grafts may facilitate bone and/or cartilage repair, promote bone and/or cartilage growth, and/or or promote bone and/or cartilage regeneration at the defect site/implant site in the subject. Exemplary graft shapes for use to treat such defects are shown, or readily apparent from, FIGS. 4B-4D. In some instances, the graft shape is selected, or is shaped, to fit (be complementary to) the configuration of the defect site.

In some embodiments, the methods provided may include administering a composite graft to treat a subject having a muscle defect. A graft may be administered to a subject to repair, augment, or replace a muscle, or promote muscle growth and/or regeneration, in the subject. In some instances, the muscle defect may be a degenerative defect or injury. In some instances, the muscle defect may be a traumatic defect or injury. In some instances, methods of treating muscle defects may be reconstructive. For example, a graft may be implanted a defect site/implantation site at which the native muscle tissue is fully or partially missing. For example, due to disease or injury, a muscle may be damaged, missing, or removed in a leg, an arm, a chest (including a breast), a back, or a face. Exemplary graft shapes for use to treat defects in a leg or arm are shown, or readily apparent from, FIG. 5. In some instances, the methods are for treatment (repair or reconstruction) of degenerated, broken, or missing soft tissue from the oral (mouth) and maxillofacial (jaws and face) region of a subject. In some instances, the grafts are selected, or are shaped, to mimic the configuration of the missing native muscle tissue. The methods may include administering a vulnerary composite graft to a patient with a muscle defect, the vulnerary composite graft containing a vulnerary biological component. In some instances, the composite graft may facilitate muscle repair, promote muscle growth, and/or or promote muscle regeneration at the defect site/implant site in the subject. In some instances, vulnerary biological components such as mesenchymal stem cells can migrate out of the implanted graft and carry out repair and regeneration functions. For example, the mesenchymal stem cells can reproduce and form new muscle. The newly established muscle cell population can fill defects and integrate with existing native muscle tissue at the implantation site. In this way, vulnerary composite grafts that are implanted at a defect site within a patient may treat muscle defects. In some instances, the vulnerary composite grafts may be bioresorbable by virtue of the bioresorbable synthetic scaffold. Such grafts may be absorbed by the subject's body over time as the vulnerary biological component facilitates healing of the muscle defect.

In some embodiments, the methods provided may include administering a composite graft to treat a subject having a skin defect. In some embodiments, the implant may be administered to a subject to repair skin, promote skin growth, and/or skin regeneration in the subject. In some instances, the skin defect may be a degenerative defect or injury. In some instances, the skin defect may be a traumatic defect or injury. For example, the skin defect may be a burn. In another example, the skin defect may be an abrasion or abraded region of skin. In another example, the skin defect may be a region from which a melanoma has been removed. Exemplary graft shapes for use to treat such defects are shown, or readily apparent from, FIG. 6A-6B. The methods may include administering a vulnerary composite graft to a patient with a skin defect, the vulnerary composite graft containing a vulnerary biological component. In some instances, the composite graft may facilitate skin repair, promote skin growth, and/or or promote skin regeneration at the defect site/implant site in the subject. In some instances, vulnerary biological components such as mesenchymal stem cells or keratinocytes can migrate out of the implanted graft and carry out repair and regeneration functions. The newly established skin cell population can fill defects and integrate with existing native skin at the implantation site. In this way, vulnerary composite grafts that are implanted at a defect site within a patient may treat skin defects. In some instances, the vulnerary composite grafts may be bioresorbable by virtue of the bioresorbable synthetic scaffold. Such grafts may be absorbed by the subject's body over time as the vulnerary biological component facilitates healing of the skin defect.

### IV. Methods and Systems of Manufacturing

Provided in this disclosure are also method and systems for manufacturing the composite grafts described above.

In one aspect, provided are systems useful for manufacturing composite grafts of the disclosure. The systems include various components. As used herein, the term “component” is broadly defined and includes any suitable apparatus or collections of apparatuses suitable for carrying out the manufacturing methods described herein. The components need not be integrally connected or situated with respect to each other in any particular way. Embodiments include any suitable arrangements of the components with respect to each other. For example, the components need not be in the same room. However, in some instances, the components are connected to each other in an integral unit. In some instances, the same components may perform multiple functions.

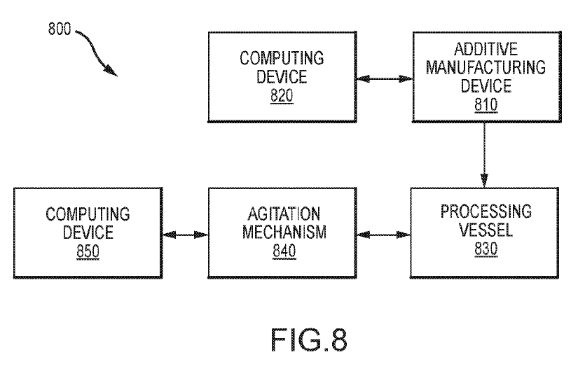

Turning to the drawings, FIG. 8 depicts a schematic of representative system 800 for manufacturing the composite grafts described herein. In some embodiments one or more components shown in FIG. 8 may be omitted. Similarly, in some embodiments, components not shown in FIG. 8 may also be included.

The system 800 may include an additive manufacturing device 810. Additive manufacturing devices generally use one or more substrate dispensing or writing elements that move in a plane, deposit substrate, and (optionally) cure substrate. Additional motion by the manufacturing device mechanism, generally perpendicular to the plane of the added substrate layers, enables the device to write/add layer after layer, gradually adding physical details to construct a solid, three dimensional synthetic scaffold out of non-solid substrate. The successive layers of material are generally deposited under computer control. The time required to build a synthetic scaffold depends on various parameters, including the speed of adding a layer of the synthetic substrate, the solidification/curing time of the synthetic substrate, the intensity of the curing agent (if any), and the desired resolution of the scaffold details. As described further with respect to the manufacturing method, the additive manufacturing device 810 may be capable of performing at least one type of additive manufacturing process to manufacture the synthetic scaffolds described herein.

In one aspect, the system 800 may include a processing vessel 830 that is configured to receive the scaffold. The processing vessel 830 is of sufficient size to contain a desired volume of processing fluid. Generally, the processing vessel 830 may be made of a non-reactive plastic or resin, metal, or glass. In some instances, the processing vessel 830 may be a beaker, flask, test tube, conical tube, bottle, vial, dish, or other vessel suitable for containing the scaffold and the processing fluid in a sealed environment.

In another aspect, the system 800 includes an agitation mechanism 840. In some instances, the agitation mechanism 840 is a resonant acoustic vibration device that applies resonance acoustic energy to the processing vessel and its contents. Low frequency, high-intensity acoustic energy may be used to create a uniform shear field throughout the entire processing vessel, which results in rapid fluidization (like a fluidized bed) and dispersion of material. The resonant acoustic vibration device introduces acoustic energy into the processing fluid contained by the processing vessell 830 and the graft components therein. In some instances, the resonant acoustic vibration device includes an oscillating mechanical driver that create motion in a mechanical system comprised of engineered plates, eccentric weights and springs. The energy generated by the device is then acoustically transferred to the material to be mixed. The underlying technology principle of the resonant acoustic vibration device is that it operates at resonance. An exemplary resonant acoustic vibration device is a Resodyn LabRAM ResonantAcoustic© Mixer (Resodyn Acoustic Mixers, Inc., Butte, Mont.). In some instances, the resonant acoustic vibration device may be devices such as those described in U.S. Pat. No. 7,866,878 and U.S. Patent Application Nos. 20150146496 and 20160236162. In other embodiments, the agitation mechanism 840 may be shaker, mechanical impeller mixer, ultrasonic mixer, sonicator, or other high intensity mixing device.

Resonant acoustic mixing by such resonant acoustic vibration devices as described above is a non-contact mixing technology that relies upon the application of a low-frequency acoustic field to facilitate mixing. Resonant acoustic mixing works on the principle of creating micro-mixing zones throughout the entire mixing vessel, which provides faster, more uniform mixing throughout the processing vessel than can be created by conventional, state-of-the-art mixing systems. Resonant acoustic mixing differs from conventional mixing technology where mixing is localized at the tips of the impeller blades, at discrete locations along the baffles, or by co-mingling products induced by tumbling materials. A resonant acoustic vibration device as described herein does not require impellers, or other intrusive devices to mix, nor does it require unique processing vessel designs.

A resonant acoustic vibration device as described herein operates at mechanical resonance, resulting in a virtually lossless transfer of the device's mechanical energy into the materials being mixed in the processing vessel created by the propagation of an acoustic pressure wave in the mixing vessel. In contrast, conventional mechanical mixers are typically designed to specifically avoid operating at resonance, as this condition can quickly cause violent motions and even lead to catastrophic failure of the system. However, in the resonant acoustic vibration device contemplated herein, operation at resonance enables even small periodic driving forces to produce large amplitude vibrations that are harnessed to produce useful work. Such devices store vibrational energy by balancing kinetic and potential energy in a controlled resonant operating condition. The resonant frequency of such systems is the frequency at which the mechanical energy in the device can be perfectly transferred between potential energy stored in the springs of such a device and the kinetic energy in the moving masses therein when the device is in operation.

Resonant acoustic vibration devices as described herein may be a three-mass system comprising multiple masses (such as plates), a spring assembly system, and the processing vessel that are simultaneously moving during mixing. The springs store potential when an applied external force compresses or stretches the spring, with the stored energy proportional to the degree to which the spring is distorted. Such devices comprise a damper that absorbs energy when the device/system is in motion. The formula below desires the forces present during oscillation in the resonant acoustic vibration device:

$${( m \cdot ( \frac{{d}^{2}}{{dt}^{2}} ) x ( t ) )}_{I} + {( c \cdot ( \frac{d}{dt} ) x ( t ) )}_{II} + {k \cdot x ( t )}_{III} = {{F}_{o} \cdot sin ( {ω}_{f} \cdot t )}_{IV}$$

where m is mass of the processing vessel and contents, c is the mixing constant, k is the spring rate of the spring in the device/system, F_{O} is the actual force value (input force),and ω_{f} is the actual angular frequency value of the device/system. Part I of the formula represents the inertia forces in the device/system, part II represents the mixing forces in the device/system, part III represents the stored forces in the device/system, and part IV represents the input forces in the device/system. The inertia forces are represented by the inertial component of the system, mass. The forces when oscillating include the damping (mixing) forces and the stored (spring) forces. This formula shows the relationship between the forces due to the moving masses, the deflected springs, and the mixing process. As shown in the formula, these forces sum to be equal to the mechanical force driving the system. The resonant acoustic vibration devices described herein may comprise softward that automatically senses the system resonance condition, and adjusts the operating frequency to maintain resonance throughout the mixing process, even when state changes in the contents of the processing vessel cause the coupling and damping characteristics of the contents to change.

At a particular oscillation frequency, the resonant frequency, the stored forces in the springs are directly offset by the inertia forces of the masses (plates and processing vessel), and cancel over one period of oscillation. Thus, the device/system can oscillate without the need for charging the spring or providing energy to the mass during the cycles. For frequencies below resonance, energy is lost in charging the springs and, for frequencies above resonance, energy has to be added to maintain the inertial energy. The result of operating at resonance, is that the amplitude of the oscillations reaches a maximum, while the power required is at a minimum. The power consumed by the system is transferred directly into the contents of the processing vessel.

In one embodiment, the resonant acoustic vibration devices as described in U.S. Pat. No. 7,866,878 and U.S. Patent Application Nos. 20150146496 and 20160236162 operate at mechanical resonance, which is nominally 60 Hz. The exact frequency of mechanical resonance during mixing by the resonant acoustic vibration devices described herein is only affected by the processing vessel (and its contents), the equivalent mass, and how well the contents couple to the processing vessel and absorb energy as motivated.

Resonant acoustic mixing by such resonant acoustic vibration devices as described above can be performed on low viscosity liquids, high viscosity liquids, non-Neutonian fluids, solid materials, and combinations thereof. For example, liquids in a processing vessel that is being subjected to a low-frequency acoustic field in the axial direction resulting in second order bulk motion of the fluid, known as acoustic streaming, which are rotational currents circulating between the top and the bottom of the fluid in the processing vessel. This in turn causes a multitude of micro-mixing cells (micro-circular currents) throughout the vessel. Typically, the characteristic mixing lengths (diameters) for such micro-mixing cells is about 50 microns when the resonant acoustic vibration device is operating at 60 Hz. The strength of the pressure waves associated with the acoustic streaming flow is strongly correlated to the displacement of the acoustic source (the base of the processing vessel). In another example, when solids are mixed in the processing vessel, mixing is based on collisions. Solids in the processing vessel are excited by collisions with the vessel base and collisions with other particles in the vessel that can result in harmonic vibrations of the vessel with the solid contents therein (particularly particles). The particle motions are dependent upon the vibration amplitude, A, frequency, ω), and the resultant accelerations that the particles undergo. The chaotic motions created within the processing vessel by the resonant acoustic vibration devices cause a great degree of particle-to-particle disorder, microcell mixing, as well as creating bulk mixing flow. Regardless of the contents being mixed in the processing vessel, the resonant acoustic vibration device uses an acoustic field to provide energy into the contents being mixed in a manner that is uniform throughout the mixing container, rather than at discrete locations, or zones in the mixing vessel, as is accomplished by most state-of-the-art mixing technologies.

The system 800 may comprise one or more computing devices such as, for example, computing devices 820 and 850. Typical examples of computing devices 820 and 850 include a general-purpose computer, a programmed microprocessor, a microcontroller, a peripheral integrated circuit element, and other devices or arrangements of devices that are capable of implementing the steps that constitute the provided manufacturing processes. The computing devices 820 and 850 may comprise a memory and a processor. In some instances, the memory may comprise software instructions configured to cause the processor to execute one or more functions. The computing devices can also include network components. The network components allow the computing devices to connect to one or more networks and/or other databases through an I/O interface.

For computing device 820, the software instructions may be configured to cause the processor to coordinate the components of the additive manufacturing device 810 to form the synthetic scaffold from a synthetic material and a biological component. For example, the software instructions may include a timed and/or sequential addition of the synthetic material an, optionally, one or more other reagents into the desired configuration of the synthetic scaffold. The software instructions may include a timed and/or sequential increase or decrease in temperature of the synthetic material and/or other reagents in the additive manufacturing process. In another example the software instruction may cause timed and/or sequential physical, mechanical, or electrochemical adjustment to the components of the additive manufacturing device 810 to effect the additive manufacturing process. In some instances, the memory may comprise software instructions configured to perform any aspect of the additive manufacturing process within the scope of this disclosure. In some instances, computing device 820 may be configured as part of the additive manufacturing device 810. In another instance, computing device 820 may be separate from but in communication with the additive manufacturing device 810.

For computing device 850, the software instructions may be configured to cause the processor to coordinate the components of the agitation mechanism 840 to agitate the processing vessel 830 and its contents. For example, the software instruction may cause timed and/or sequential physical, mechanical, or electrochemical adjustment to the components of the agitation mechanism 840 to agitate the processing vessel 830 for one or more periods of time, at one or more agitation speeds, or a combination thereof. In one example, where the agitation mechanism 840 is a resonant acoustic vibration device, the software instructions may include a timed and/or sequential application of resonant acoustic energy of a selected intensity and a selected frequency for a selected period of time. The software instructions may have a range of parameter settings for selection depending on the nature of the scaffold, the biological component, the processing fluid, or a combination thereof. In some instances, computing device 850 may be configured as part of the agitation mechanism 840. In another instance, computing device 850 may be separate from but in communication with the agitation mechanism 840.

In some instances, systems of the disclosure include all of the components of system 800. For example, system 800 in its entirety is useful for manufacturing composite grafts that include a synthetic scaffold printed together with a first biological component and subsequently combined with a second biological component via agitation. In other instances, systems of the disclosure may include only some of the components of the system 800. For example, a system comprising additive manufacturing device 810, and, optionally, computing device 850 is useful for manufacturing composite grafts that include a synthetic scaffold printed together with a first biological component. It is contemplated that the systems of the disclosure may also include other components that facilitate the additive manufacturing process or the mixing of the biological component with the scaffold to form the composite graft.

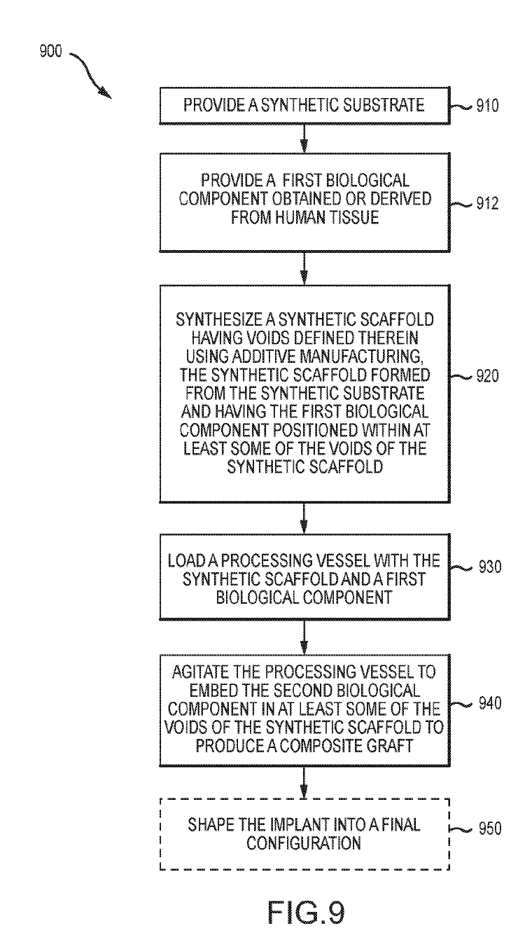

In another aspect, provided are methods for manufacturing composite grafts of the disclosure. Exemplary method 900 is shown in FIG. 9 and described below. The steps of the method is described below with reference to components described above with regard to system 800 as shown in FIG. 8. In some embodiments, one or more steps shown in FIG. 9 may be omitted or performed in a different order. Similarly, in some embodiments, additional steps not shown in FIG. 9 may also be performed.

FIG. 9 is a flow chart of steps for performing a method 900 of manufacturing a composite graft having a synthetic scaffold according to one embodiment. The method 900 begins at step 910 with providing a synthetic substrate from which the synthetic scaffold is to be synthesized. The synthetic substrate 910 may be a bioresorbable polymer. In some instances, the bioresorbable polymer may include polylactides, polyglycolides, polyanhydrides, polycaprolactones, oxidized cellulose, alginate polymers or derivative thereof, fibrin polymers or derivatives thereof, or copolymers of any combination thereof. In some instances, the synthetic substrate may have been integrated with cellular adhesion molecules that support the physical attachment of cells. In some instances, the synthetic substrate may have structural integrity sufficient to maintain the physical properties of the composite graft and also be receptive to cellular proliferation and integration. The synthetic substrate is selected based on the desired physical properties of the composite graft as described above. In some instances, the type of synthetic substrate selected may influence the quality of the composite graft in terms of, for example, any of degree of flexibility (hardness), strength, and compressibility. An advantage of using a bioresorbable polymer as the synthetic substrate is that the additive manufacturing process may be used to print a composite graft having a synthetic scaffold and the biological component positioned in at least some of the voids defined therein. Generally, a bioresorbable polymer has a melting temperature not greater than 50° C. This low melting temperature may permit the biological component to be combined with the synthetic substrate during printing without negatively impacting the biological or therapeutic activity thereof.

Method 900 the proceeds with step 912 in which a first biological component is provided. The first biological component is selected based on the intended type of composite graft to be manufactured by the method. For example, an osteogenic biological component is selected if the product of the manufacturing method is to be an osteogenic composite graft. In another example, a chondrogenic biological component is selected if the product of the manufacturing method is to be a chondrogenic composite graft. In another example, at least one of a osteogenic biological component and a chondrogenic biological component is selected if the product of the manufacturing method is to be an osteochondral composite graft. In another example, a vulnerary biological component is selected if the product of the manufacturing method is to be a vulnerary composite graft. In some instances, step 912 includes providing more than one type of biological component.

Once the synthetic substrate and biological component are selected, the synthetic scaffold of the composite graft can be fabricated through an additive manufacturing process (also referred to as printing herein) using additive manufacturing device 810 according to step 920 of method 900. Additive manufacturing device 840 fabricates the synthetic scaffold to have a trabecular configuration (a plurality of voids in a least a portion of the scaffold) and the first biological component positioned in at least a portion of the voids of the scaffold. In some instances, the synthetic scaffold is synthesized to have desired shape and dimensions of the composite graft. In some instances, the trabecular configuration of the synthetic scaffold is selected based on the properties of the biological component integrated therein, the desired end purpose (use) of the graft, or both. In some instances, the synthetic scaffold is printed to have voids defined therein that are relatively uniform in size and shape. In some instances, the synthetic scaffold is printed to have voids of various sizes or shapes (or both) defined therein. In some instances, a first portion of the scaffold may have voids of a first size and a second portion of the scaffold may have voids of a different size. In some instances, the composite graft may be synthesized with more than one type of biological component. In some instances, the first biological component may be uniformly positioned in the voids defined in the scaffold or may be positioned in only a portion of the voids. In some instances, the scaffold may have voids of different sizes and or shapes. In such instances, voids of different sizes/shapes may accommodate different biological components in different portions of the graft. For example, an osteochondral graft may have a bone-facing, or bone-contacting, portion, and a cartilage-facing, or cartilage-contacting portion (see, for example, FIG. 4C). In some instances, the bone-contacting portion of the grafts may have an osteogenic biological component positioned within voids defined therein and the cartilage-contacting portion of osteochondral grafts may have a chondrogenic biological component positioned within voids defined therein. In some instances, during printing step 920, different biological components may be printed into the voids of distinct portions of the synthetic scaffold. As discussed above, software instructions on computing device 850 may include detailed configuration instructions for synthesis of the composite graft.

In some instances, the composite graft may be synthesized in the shape of a bone or portion of a bone. For example, the composite graft may be synthesized in the shape of a long bone, or portion thereof, as depicted in FIG. 2A and FIG. 2J. In another example, the composite graft may be synthesized in the shape of a facial bone, a skull bone, or a portion of either, as depicted in FIG. 2B. In another example, the composite graft could be synthesized in the shape of a jaw bone, or portion thereof, as depicted in any of FIGS. 2C-2E. In some instances, the composite graft may be synthesized in the shape of an intervertebral disc, exemplary structures thereof as shown in FIG. 2F and FIG. 21. In some instances, the composite graft may be synthesized in the shape of a nasal implant. For example, the composite graft may be synthesized in the shape of cartilage found in a nose, or a portion thereof, as depicted in FIG. 3A. In some instances, the composite graft may be synthesized in the shape of an ear, or portions thereof, exemplary structures of which are shown in FIGS. 3B-3C. In some instances, the composite graft may be synthesized in the shape of a cartilage patch, exemplary structures of which are shown in FIG. 4A and FIG. 4D. In some instances, the composite graft may be synthesized in the shape of an osteochondral plug, exemplary structures of which are shown in FIG. 4C and FIG. 4D. In some instances, the composite graft may be synthesized in the shape of a muscle, exemplary structures of which are shown in FIG. 5. In some instances, the composite graft may be synthesized in the shape of a skin patch, exemplary structures of which are shown in FIGS. 6A-6B. In some instances, the composite graft may be in the shape of a cube, strut, or strip, such as shown in FIG. 1D.

Various additive manufacturing methods may be used to fabricate the synthetic scaffold. In some instances, the additive manufacturing process may be drop-based bioprinting. Drop-based bioprinting creates composite grafts using individual droplets of a synthetic substrate, which may be combined with a biological component (such as those described in this disclosure). Upon contact with a substrate surface, each droplet begins to polymerize, forming a larger structure as individual droplets coalesce. Polymerization is instigated by the presence of calcium ions on the substrate, which diffuse into the liquified bioink and allow for the formation of a solid gel. This process may be efficient in terms of speed. In some instances, the additive manufacturing process may be extrusion bioprinting. Extrusion bioprinting involves the constant deposition of a synthetic substrate and biological component from an extruder, a type of mobile print head. This process may permit controlled and gentle biological component deposition. In some instances, this process may permit greater biological component density in the composite graft. In some instances, extrusion bioprinting may becoupled with UV light, which photopolymerizes the synthetic substrate to form a more stable, integrated composite graft. The type of additive manufacturing process selected for method 900a may depend on the type of synthetic substrate selected, the desired physical properties of the composite graft, or both.

When the synthetic substrate selected is a polymer, the additive manufacturing process may involve polymerization of polymer to form the synthetic scaffold. Polymerization causes a polymerizing agent (polymer) to cure (harden/solidify). Some polymerizing agents can self-polymerize without the addition of any addition agents, such as in response to time, temperature change, or other change in environmental factor, or a combination thereof. An exemplary self-polymerizing agent is polyethylene. In some instances, a polymerizing agent may be combined with one or more hardening agents to facilitate polymerization (curing). A hardening agent may be a cross-linker or cross-linking agent. In some instances, a polymer may require the addition of one or more softening agents. For example, a synthetic scaffold used as an implant to replace a muscle may require the addition of a softening agent. Detailed discussion of polymers, including aspects of polymerization and features thereof, is provided in U.S. patent application Ser. No. 14/923,087, filed Oct. 26, 2015, the contents of which is incorporated herein in its entirety for all purposes.

In some instances, a biological adhesive may be combined with the synthetic substrate, the first biological component, or both, before or during the additive manufacturing process. In some instances, the biological adhesive may be printed onto at least a portion of the synthetic scaffold (such as in the voids defined therein) during the additive manufacturing process. In such instances, the biological component may be printed onto the biological adhesive.

In some instances, the additive manufacturing process of step 920 yields a composite graft of this disclosure. As described further below, the composite graft may further shaped into a final configuration in step 950. In some instances, as described further below, method 900 may also include combining the composite graft with a biocompatible solution. In certain instances, as described further below, method 900 may also include combining the composite graft with an additional biological component. Before discussing these steps, an alternative manufacturing method including steps 930 and 940 will be described. In this alternative manufacturing method, the composite graft synthesized in step 920 may be considered a partial composite graft.

In some instances, the method 900 may further continue with step 930, which involves loading the synthetic scaffold into processing vessel 830 with a second biological component. In some instances, the second biological component comprises particulates that are relatively uniform in size and shape as shown in FIG. 1B. In some instances, the second biological component comprises particulates that have different shapes and sizes as shown in FIG. 1C. In some instances, the first biological component and the second biological component comprise particulates that are relatively similar in size and dimension. In some instances, the first biological component and the second biological component comprise particulates that have different shapes and sizes. In some instances, the composite graft synthesized in step 920 may include voids configured to contain the first biological component and voids configured to contain the second biological component.

The processing vessel 830, as discussed above, is configured to receive the scaffold and is of sufficient size to contain a desired volume of processing fluid, the processing fluid containing the first biological component. The processing fluid may be a biocompatible solution. In some instances, the biocompatible solution may be a buffered solution, a nutritive media, or a cryopreservation medium. The nutritive medium may be a a growth medium. Exemplary buffered solutions include phosphate buffer saline, MOPS, HEPES, and sodium bicarbonate. The pH of the solution is generally in the range of pH 6.4 to 8.3. Suitable examples of growth medium include, but are not limited to, Dulbecco's Modified Eagle's Medium (DMEM) with 5% Fetal Bovine Serum (FBS). In some instances, growth medium may include high glucose DMEM. Cryopreservative medium may include one or more cryoprotective agents such as, but not limited to, glycerol, DMSO, hydroxyethyl starch, polyethylene glycol, propanediol, ethylene glycol, butanediol, or polyvinylpyrrolidone. In one example, a cryopreservation medium may include DMSO and glycerol. In some instances, the biocompatible solution may include an antibiotic.

Method 900 may then proceed to step 940 to produce the composite graft having the second biological component embedded therein. Step 940 involves agitating the processing vessel containing the synthetic scaffold and the first biological component so as to embed the second biological component in at least some of the voids of the synthetic scaffold and produce the composite graft. This step is performed using agitation mechanism 840, which, as discussed above, may be a resonant acoustic vibration device, a shaker, a mechanical impeller mixer, an ultrasonic mixer, a sonicator, or other high intensity mixing device.

In some instances, the second biological component may be uniformly positioned in the voids defined in the scaffold or may be positioned in only a portion of the voids. In some instances, the scaffold may have voids of different sizes and or shapes. In such instances, voids of different sizes/shapes may accommodate different biological components in different portions of the graft. For example, an osteochondral graft may have a bone-facing, or bone-contacting, portion, and a cartilage-facing, or cartilage-contacting portion (see, for example, FIG. 4C). In some instances, the bone-contacting portion of the grafts may have an osteogenic biological component positioned within voids defined therein and the cartilage-contacting portion of osteochondral grafts may have a chondrogenic biological component positioned within voids defined therein. In some instances, the second biological component (and, optionally, additional biological components) may be embedded into voids located in one or more distinct portions of the partial composite graft due to the size or shape (or both) of the voids.

In some instances, the agitating step may be performed using a resonant acoustic vibration device as the agitation mechanism 840 to agitate the processing vessel and its contents using resonant acoustic vibration. According to some embodiments, resonant acoustic vibration applies low acoustic frequencies and high energy to a mechanical system of the resonant acoustic vibration device, which in turn is acoustically transferred to processing vessel 830 positioned within the resonant acoustic vibration device. The mechanical system operates at resonance and, as such. there is near-complete exchange of energy from the mechanical system to the contents of the processing vessel. In some instances, only the contents of the processing vessel 830 absorb energy generated by the resonant acoustic vibration device. In some instances, the acoustic energy generated by may create a uniform shear field throughout the processing vessel 830, resulting in rapid dispersion of the biological components in the processing fluid in the processing vessel. In some instances, acoustic energy may introduce multiple small scale intertwining eddies throughout the processing fluid in the processing vessel 830. As compared with mechanical impeller agitation, resonant acoustic vibration mixes by creating microscale turbulence, rather than mixing through bulk fluid flow. Similarly, as compared with ultrasonic agitation (sonication), resonant acoustic vibration uses magnitudes lower frequency of acoustic energy and enables a larger scale of mixing.

In some instances, the agitating step may include applying resonant acoustic vibration having an acoustic frequency in the range of 15 Hertz and 60 Hertz to the processing vessel. In certain instances, acceleration of the acoustic resonance vibration may be in the range of 10 to 100 times the energy of g-force. In some instances, the acceleration of the acoustic energy vibration may be in the range of 40 to 60 times the energy of g-force. G-force refers to either the force of gravity on a particular extraterrestrial body or the force of acceleration anywhere. In the context of this disclosure, g-force refers to the force of acceleration produced by a resonant acoustic vibration device. The unit of g-force is “g”, where 1 g is equal to the force of gravity at the Earth's surface, which is 9.8 meters per second per second. The frequency or the energy of the resonant acoustic vibration, or both, may be selected so as to minimize deleterious effects on the first biological component (for example, cell lysis, protein denaturation, etc.).

The agitation step 940 is performed for sufficient time to cause a desired amount of the first biological component to embed in the voids of the synthetic scaffold. In some instances, the agitation time may be selected so as to minimize deleterious effects on the first biological component (for example, cell lysis, protein denaturation, etc.). Exemplary agitation periods include 5 minutes, 10 minutes, or 30 minutes. In some instances, the agitation time may comprise a single period of time during which agitation is continuously applied. In other instances, the agitation time may comprise discontinuous periods of agitation. For example, the duration of time of agitation may be repeated in a number of cycles from one to five.

During the agitation step 940, the temperature of the contents in the processing vessel 830 are kept within an acceptable range. For example, the temperature may be maintained between 15° C. and 40° C. The temperature of the processing vessel 830 may be selected so as to minimize deleterious effects on the first biological component (for example, cell lysis, protein denaturation, etc.).

In some instances, the composite graft produced by agitation step 940 may be assessed to determine the amount of biological component that has been embedded in the scaffold. In some instances, this may be performed by assessing a change in weight of the scaffold before and after agitation step 940. In some instances, this may be performed by staining the composite graft with a reagent that identifies the biological component. In some instances, this may be performed by assessing a change in concentration of the biological component in the processing fluid before and after agitation step 940.

In some instances, a biological adhesive may be combined with the second biological component, the partial composite graft, or both, in the processing vessel 830. For example, the partial composite graft may be combined with the adhesive and then placed in the processing vessel 830. In another example, the second biological component may be combined with the adhesive prior to or after being placed in the processing vessel 830. In some instances, the adhesive is added to processing vessel 830 with the partial composite graft and second biological component.

In this alternative manufacturing method in which steps 910, 912, 920, 930, and 940 of method 900 have been performed, the manufactured composite graft include a synthetic scaffold having a trabecular structure in which a first biological component is positioned within a portion of the voids defined in the scaffold and a second biological component is positioned within a portion of the voids defined in the scaffold.

The following steps may be performed on either of the composite graft produced by steps 910, 912, and 920 or the composite graft produced by steps 910, 912, 920, 930, and 940.

Method 900 may optionally proceed to step 950 in which the composite graft produced in agitation step 940 is shaped into a final configuration. In some instances, the composite graft may be shaped prior to packaging by the manufacturer. In some instances, the composite graft may be shaped by a medical professional to be compatible with the configuration and/or dimensions of the implantation site. It is contemplated that the implant may be shaped such as by cutting, bending, folding, grinding, drilling, and the like. For example, the composite graft may be shaped with a surgical tool, such as a scalpel or scissors, a mechanical blade, or a laser. In some instances, the composite graft may be shaped into a final configuration to fit a patient's unique needs due to the variations in their activity level, anatomy, disease, and/or trauma. In some instances, the shaping will occur prior to implantation in the patient. In some instances, the shaping will occur during implantation in the patient (intraoperatively).

In some instances, method 900 may further include combining the composite graft with a biocompatible solution. In some instances, the biocompatible solution may be a buffered solution, a nutritive media, or a cryopreservation medium. The nutritive medium may be a growth medium. Exemplary buffered solutions include phosphate buffer saline. Suitable examples of growth medium include, but are not limited to, Dulbecco's Modified Eagle's Medium (DMEM) with 5% Fetal Bovine Serum (FBS). In some instances, growth medium may include high glucose DMEM. Cryopreservative medium may include one or more cryoprotective agents such as, but not limited to, glycerol, DMSO, hydroxyethyl starch, polyethylene glycol, propanediol, ethylene glycol, butanediol, or polyvinylpyrrolidone. In one example, a cryopreservation medium may include DMSO and glycerol. In some instances, the biocompatible solution may include an antibiotic.

In some instances, method 900 may further include combining the composite graft an additional biological component. In some instances, the biological component may include tissue particles. In some instances, the biological component may include growth factors. In some instances, the biological component may include cells. In some instances, the biological component may include platelet-rich plasma (PRP). In some instances, the biological component may include a combination of two or more of tissue particles, growth factors, PRP, and cells.

In some instances, the composite grafts may be stored at room temperature, refrigerated (approximately 5-8° C.), or frozen (approximately −20° C., −80° C., −120° C.)

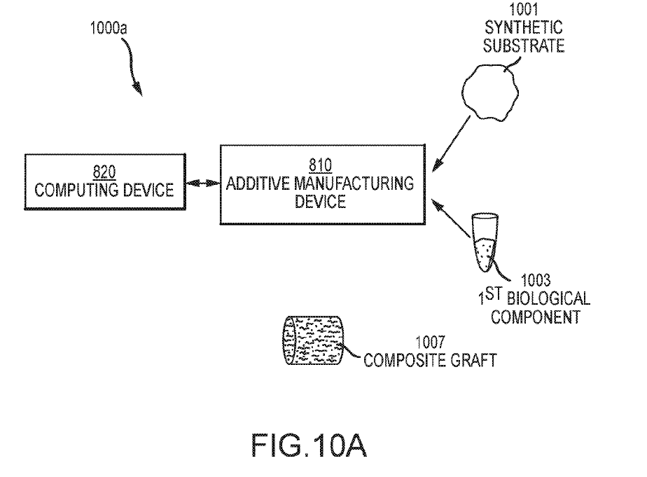

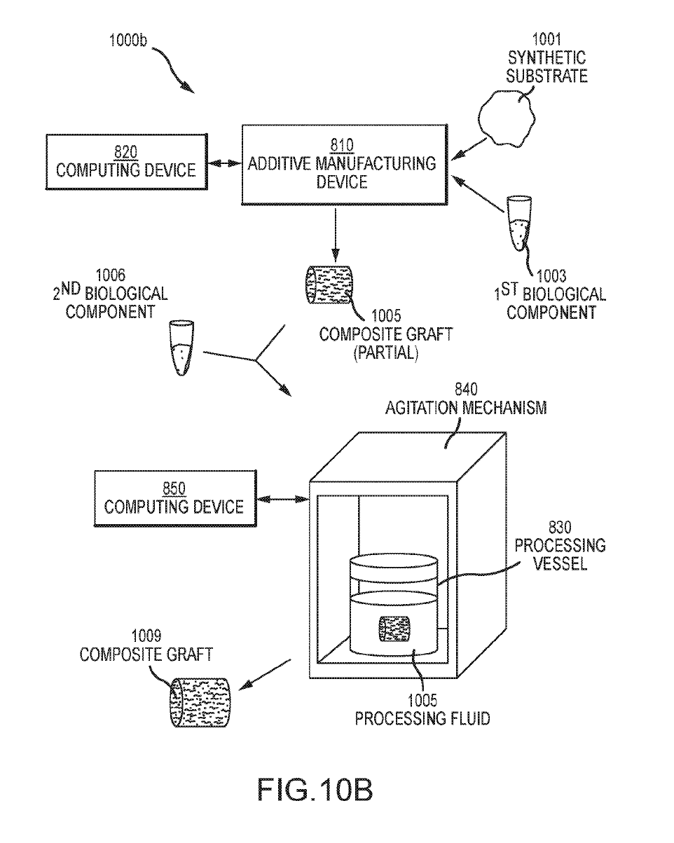

To further illustrate the methods and systems of this disclosure, example methods according to method 900 as performed on system 800 are depicted graphically in FIG. 10A and FIG. 10B. FIG. 10A describes a method comprising steps 910, 912, and 920 of method 900, which FIG. 10B describes a method comprising steps 910, 912, 920, 930, and 940 of method 900. Both FIG. 10A and FIG. 10B make reference to the components of system 800 as described above. In FIG. 10A and FIG. 10B, the synthetic scaffold 1001 and composites grafts 1006 and 1008 may be any of the synthetic scaffolds and composite grafts, respectively, described above in this disclosure, including those depicted in, or described with respect to, FIG. 1B, FIG. 1C, FIG. 1E, FIGS. 2A-2J, FIGS. 3A-3C, FIGS. 4A-4D, FIG. 5, and FIGS. 6A-6B. Similarly, first biological component 1003 and second biological component 1006 of FIG. 10A and FIG. 10B may be any of the biological components described above in this disclosure, including those depicted in, or described with respect to, FIGS. 1A-1D.

As shown in FIG. 10A, synthetic substrate 1001 and first biological component 1003 are provided according to steps 910 and 912 and synthesized into composite graft 1007 using additive manufacturing device 810 according to step 920. Computing device 820 may control the additive manufacturing process performed by additive manufacturing device 810 to synthesize composite graft 1007 having a trabecular structure comprising voids defined in the scaffold 1004 and the first biological component 1003 positioned in at least a portion of the voids. Composite graft 1007 generally has the shape and dimensions desired for the final composite graft. Composite graft 1007 may further be processed/shaped into a final configuration if desired by the manufacturer or user.

As shown in FIG. 10B, synthetic substrate 1001 and first biological component 1003 are provided according to steps 910 and 912 and synthesized into partial composite graft 1005 using additive manufacturing device 810 according to step 920. Computing device 820 may control the additive manufacturing process performed by additive manufacturing device 810 to synthesize partial composite graft 1005 having a trabecular structure comprising voids defined in the scaffold 1004 and the first biological component 1003 positioned in at least a portion of the voids. In some instances, partial composite graft 1005 may have the shape and dimensions desired for the final composite graft. The partial composite graft 1005 is combined with the second biological component 1006 in processing fluid 1005, all of which are disposed in processing vessel 830 according to step 930. Processing vessel 830 is then positioned in, or on, agitation mechanism 840 and agitated according to step 940 to embed the second biological component 1006 into at least a portion of the voids of partial composite graft 1005, thereby producing composite graft 1009. In some instances, agitation mechanism 840 is an acoustic resonant vibration device and the processing vessel 830 is placed inside of the device. Computing device 850 may control the operation of agitation mechanism 840, determining the energy and duration of the agitation period. Agitation mechanism 840 may also be maintained at a controlled temperature (ambiently or internally, or both) to maintain the temperature of processing vessel 830 and its contents within a desired range. Composite graft 1009 may further be processed/shaped into a final configuration if desired by the manufacturer or user.

All features of the described systems are applicable to the described methods mutatis mutandis, and vice versa.

All patents, patent publications, patent applications, journal articles, books, technical references, and the like discussed in the instant disclosure are incorporated herein by reference in their entirety for all purposes.

It is to be understood that the figures and descriptions of the disclosure have been simplified to illustrate elements that are relevant for a clear understanding of the disclosure. It should be appreciated that the figures are presented for illustrative purposes and not as construction drawings. Omitted details and modifications or alternative embodiments are within the purview of persons of ordinary skill in the art.

It can be appreciated that, in certain aspects of the disclosure, a single component may be replaced by multiple components, and multiple components may be replaced by a single component, to provide an element or structure or to perform a given function or functions. Except where such substitution would not be operative to practice certain embodiments, such substitution is considered within the scope of the disclosure.

The examples presented herein are intended to illustrate potential and specific implementations of the invention. It can be appreciated that the examples are intended primarily for purposes of illustration for those skilled in the art. There may be variations to these diagrams or the operations described herein without departing from the spirit of the invention. For instance, in certain cases, method steps or operations may be performed or executed in differing order, or operations may be added, deleted or modified.

Different arrangements of the components depicted in the drawings or described above, as well as components and steps not shown or described are possible. Similarly, some features and sub-combinations are useful and may be employed without reference to other features and sub-combinations. Aspects and embodiments of the invention have been described for illustrative and not restrictive purposes, and alternative embodiments will become apparent to readers of this patent. Accordingly, the present invention is not limited to the embodiments described above or depicted in the drawings, and various embodiments and modifications can be made without departing from the scope of the claims below.

While exemplary embodiments have been described in some detail, by way of example and for clarity of understanding, those of skill in the art will recognize that a variety of modification, adaptations, and changes may be employed. Hence, the scope of the present invention should be limited solely by the claims.

1. A composite graft comprising:
a synthetic scaffold comprising a bioresorbable polymer configured in a trabecular structure, the trabecular structure comprising voids defined in at least a portion of the synthetic scaffold; and
a biological component positioned in at least some of the voids of the synthetic scaffold, the biological component held into place within the voids as a result of friction present between the biological component and the synthetic scaffold;
wherein the synthetic scaffold comprises an anatomical shape resembling at least one of:
(i) a whole bone or a portion thereof comprising at least 10% of the whole bone and retaining at least some of the anatomical shape of the whole bone,
(ii) a whole muscle or a portion thereof comprising at least 10% of the whole muscle and retaining at least some of the anatomical shape of the whole muscle,
(iii) a portion of cartilage, or
(iv) a portion of skin, or
wherein the synthetic scaffold has a volume of 1 cm³ or greater.

2. The composite graft of claim 1, wherein the biological component comprises an osteogenic biological component, a chondrogenic biological component, a vulnerary biological component, or a combination of two or more thereof.

3. The composite graft of claim 2, wherein the osteogenic biological component comprises at least one of osteogenic tissue particles, osteogenic cells, or bone morphogenic proteins.

4. The composite graft of claim 3, wherein the osteogenic cells comprise at least one of mesenchymal stem cells, osteoblasts, or platelet rich plasma.

5. The composite graft of claim 2, wherein the chondrogenic biological component comprises chondrogenic tissue particles, chondrogenic cells, a chondrogenic growth factor, or a combination of two or more thereof.

6. The composite graft of claim 5, wherein the chondrogenic cells comprise at least one of mesenchymal stem cells or chondrocytes.

7. The composite graft of claim 2, wherein the vulnerary biological component comprises at least one of dermal tissue particles, muscle tissue particles, mesenchymal stem cells, keratinocytes, platelet rich plasma, dermal tissue particles seeded with mesenchymal stem cells, dermal tissue particles seeded with keratinocytes, or muscle tissue particles seeded with mesenchymal stem cells.

8. The composite graft of claim 1, wherein the biological component is recovered from a cadaveric donor.

9. The composite graft of claim 1, wherein the graft comprises a crescent shape, a wedge shape, a cylindrical shape, a spherical shape, a cubic shape, a pyramid shape, a cone shape, or an irregular shape.

10. The composite graft of claim 1, wherein the synthetic scaffold comprises an anatomical shape resembling at least one of:
(i) a whole bone or a portion thereof comprising at least 10% of the whole bone and retaining at least some of the anatomical shape of the whole bone,
(ii) a whole muscle or a portion thereof comprising at least 10% of the whole muscle and retaining at least some of the anatomical shape of the whole muscle,
(iii) a portion of cartilage, or
(iv) a portion of skin, and
wherein the synthetic scaffold has a volume of 1 cm³ or greater.

11. The composite graft of claim 1, further comprising a biological adhesive.

12. A method of treating a tissue defect in a subject, the method comprising administering to the subject the composite graft of claim 1 at the tissue defect site of the subject.

13. The method of claim 10, wherein the tissue defect comprises a degenerated or damaged spinal disc, a bone defect, an oral defect, a maxillofacial defect, a cartilage defect, an osteochondral defect, a muscle defect, or a skin defect.

14. The method of claim 10, wherein the composite graft is contacted with a saline solution, an antibiotic, blood, platelet rich plasma, or a combination of any thereof, prior to administering to the subject.

15. A method of manufacturing the composite graft of claim 1, the method comprising:
(a) providing a synthetic substrate comprising a bioresorbable polymer;
(b) providing the biological component; and
(c) forming the composite graft using an additive manufacturing process, wherein the biological component is positioned in at least some of the voids of the synthetic scaffold, and wherein at least some of the biological component is frictionally held into place within the voids.

16. The method of claim 15, wherein the biological component is mixed with the synthetic substrate prior to the additive manufacturing process.

17. The method of claim 15, wherein a biological adhesive is combined with at least one of the synthetic scaffold or the biological component prior to or during the additive manufacturing process.

18. The method of claim 15, wherein a biological adhesive is added onto at least one of the synthetic scaffold or the biological component during the additive manufacturing.

19. The method of claim 15, comprising combining the composite graft with one of a biocompatible solution or an additional biological component.

20. The method of claim 19, wherein the biocompatible solution is a buffered solution, a nutritive media or a cryopreservation medium.

21. A method of manufacturing the composite graft of claim 1, the method comprising:
(a) providing a synthetic substrate comprising a bioresorbable polymer;
(b) providing the biological component;
(c) forming a partial composite graft using an additive manufacturing process, wherein the biological component is positioned in at least some of the voids of the synthetic scaffold;
(d) providing a second biological component; and
(e) agitating the partial composite graft with the second biological component in a processing vessel to position at least a portion of the second biological component in at least some of the voids in the synthetic scaffold, at least a portion of the biological component frictionally held into place within the voids, thereby forming the composite implant.

22. The method of claim 21, wherein the combining comprises:
(i) placing the partial composite graft and the biological component into the processing vessel; and
(ii) applying resonant acoustic energy to the processing vessel, the resonant acoustic energy vibrating the processing vessel such that at least a portion of the second biological component is positioned within at least some of the voids defined in the synthetic scaffold and is frictionally held into place within the voids.

23. The method of claim 22, wherein the resonant acoustic energy is applied to the processing vessel for a period of time between 2 minutes and 4.5 hours.

24. The method of claim 22, wherein the resonant acoustic energy is applied in one or more intervals, each interval comprising a period of time.

25. The method of claim 22, comprising combining at least one of the synthetic scaffold or the biological component with a biological adhesive prior to agitating.

26. The method of claim 22, comprising combining the composite graft with at least one of a biocompatible solution or an additional biological component.

27. The method of claim 26, wherein the biocompatible solution is a buffered solution, a nutritive media, or a cryopreservation medium.
